(19) United States
(12) Patent Application Publication
(10) Pub. No.: US 2026/0327397 A1
(43) Pub. Date: Oct. 22, 2026

(54) PROCESS FOR RECYCLING OF A COLORED POLYMERIC MATERIAL INCLUDING DISCOLORATION AND CHEMICAL PROCESSING

(71) Applicant: BASF SE, Ludwigshafen am Rhein (DE)

(72) Inventors: Sina WITZEL, Ludwigshafen am Rhein (DE); Hannah Stephanie MANGOLD, Ludwigshafen am Rhein (DE); Indre THIEL, Ludwigshafen am Rhein (DE); Andreas KUNST, Ludwigshafen am Rhein (DE); Alexander TRAUT, Ludwigshafen am Rhein (DE); Achim STAMMER, Ludwigshafen am Rhein (DE)

(21) Appl. No.: 19/491,162

(22) PCT Filed: Jun. 18, 2024
(86) PCT No.: PCT/EP2024/066966

§ 371 Date: Dec. 8, 2025

(30) Foreign Application Priority Data
Jun. 19, 2023 (EP) 23180067.3

(51) Int. Cl. C08J 11/08; C07C 69/82; C08G 63/183; C08J 11/16; C08J 11/24

(52) U.S. Cl. CPC C08J 11/08; C07C 69/82; C08G 63/183; C08J 11/16; C08J 11/24; C08J 2367/02; C08J 2375/04

(57) ABSTRACT

Disclosed herein is a process for recycling of a colored polymeric material, where the colored polymeric material includes a polyalkylene terephthalate based polymer, and a colorant, the process including providing a colored polymeric material and a solvent system including gamma valerolactone (GVL); contacting the colored polymeric material with the solvent system including GVL provided under conditions allowing for colorant removal but not allowing for dissolution of the polyalkylene terephthalate based polymer, thereby obtaining a solvent system enriched in colorant and a polymeric material depleted in colorant; depolymerization or transesterification of the colorant-depleted polymeric material; thereby obtaining monomers, oligomers, or transesterification products.

Also disclosed herein are a monomer, oligomer, or transesterification product of a polyalkylene terephthalate based polymer obtained or obtainable by the process of recycling a colored polymeric material and a process including converting the monomers, oligomers, or transesterification products to obtain one or more polymer or polymer product.

A first aspect of the invention relates to a process for recycling of a colored polymeric material, wherein the colored polymeric material comprises (i) a polyalkylene terephthalate based polymer, (ii) a colorant and optionally (iii) a second polymer different from the polyalkylene terephthalate based polymer of (i) and optionally (iv) a third polymer different from the polyalkylene terephthalate based polymer of (i) and different from the second polymer of (iii), the process comprising (a) providing a colored polymeric material and a solvent system comprising gamma valerolactone (GVL); (b) contacting the colored polymeric material provided in (a) with the solvent system comprising GVL provided in (a) under conditions allowing for colorant removal but not allowing for dissolution of the polyalkylene terephthalate based polymer, thereby obtaining a solvent system, which is enriched in colorant compared to the solvent system provided, and a polymeric material, which is depleted in colorant compared to the colored polymeric material provided in (a); (c) depolymerisation or transesterification of the polymeric material, which is depleted in colorant obtained in (b); thereby obtaining monomers and/or oligomers or transesterification products. In a second aspect, the invention relates to a monomer, oligomer or transesterification product of a polyalkylene terephthalate based polymer obtained or obtainable by the process of the first aspect. A third aspect relates to a process comprising converting, preferably polymerizing, the monomers, oligomers or transesterification products of a polyalkylene terephthalate based polymer obtained from the process of the first aspect to obtain one or more polymer or polymer product. A fourth aspect relates to a process comprising converting the separated second polymer (iii) and/or the separated third polymer (iv) obtainable by or obtained from the process of the first aspect to obtain one or more monomer, polymer or polymer product.

The demand for polymeric materials has drastically increased over the last decades. However, the poor biodegradability has led to large amounts of plastic waste which is in Europe usually incinerated thereby losing valuable materials and generating huge CO₂ emissions. Even worse is landfill due to the poor biodegradability. Polymeric materials based on polyesters have been used extensively in the packaging sector, for example, in beverage packaging or food packaging. The vast majority of food and drink today is packaged within plastic bottles and containers, made from, for example, polyester materials comprising polyethylene terephthalate (PET). PET is also a main component of clothing nowadays. As these materials typically have poor biodegradability and are also still valuable products, it is desirable for these plastics to be recovered and recycled.

Although recycling processes have been adopted to convert the waste materials into new production materials, there are still many problems associated with recycling and recovery of polymeric materials. Waste packaging often includes a mixture of different polymeric materials containing also, for example, colorants. The same applies also for textiles, which also comprise a high amount of colored polymeric materials. Therefore, in order to recycle polymeric materials, it is common to separate polymeric materials based on their color and/or their composition. However, this sorting process is labor intensive and/or requires the use of sorting machines.

Regarding removal of colorants, EP 2 784 110 A1 describes the removal of organic colorants from polyethylene terephthalate (PET) flakes from shredded PET bottles by extracting the PET-flakes with ethylene glycol at ambient pressure and at the boiling temperature of the ethylene glycol. U.S. Pat. No. 10,876,240 B2 relates to decolorization of a dyed polyester with gaseous propylene glycol methyl ether. DE 2223466 A1 discloses a process for detaching substances from substrates at elevated temperature with a solvent, wherein the substrate is heated with the aid of the condensing vapor of a liquid, which is miscible with the solvent but does not dissolve the substrate and, subsequently, the heated substrate is treated with the solvent, whereby the substance is separated from the substrate.

While several processes are known for removing colorants from polyesters, these processes still suffer disadvantages: Depolymerization or transesterification of colored polymeric material delivers monomers and/or other esters which have to be extensively purified by filtrations, crystallization or distillations. This makes the process very expensive. If decoloration is not achieved, the product quality is low and cannot be re-used in high-value applications.

Furthermore, additional components such as further polymers present in a blend are interfering or cannot be removed as the colorant. Further drawbacks are the necessity to use toxic solvents and/or the need to use large quantities of solvents.

The technical problem underlying the present invention was thus the provision of an economical process for recovery of a polyester, which overcomes these disadvantages, and which especially enables on one hand a precise removal of colorants, while using comparatively small quantities of solvent, and, if present, a removal of further polymers, and on the other hand the recovery of non-degraded polyester, which then can be easily transformed into suitable monomeric building blocks.

A first aspect of the invention relates to a process for recycling of a colored polymeric material, wherein the colored polymeric material comprises (i) a polyalkylene terephthalate based polymer, (ii) a colorant and optionally (iii) a second polymer different from the polyalkylene terephthalate based polymer of (i) and optionally (iv) a third polymer different from the polyalkylene terephthalate based polymer of (i) and different from the second polymer of (iii), the process comprising
(a) providing a colored polymeric material and a solvent system;
(b) contacting the colored polymeric material provided in (a) with the solvent system provided in (a) under conditions allowing for colorant removal but not allowing for dissolution of the polyalkylene terephthalate based polymer, thereby obtaining a solvent system, which is enriched in colorant compared to the solvent system provided, and a polymeric material, which is depleted in colorant compared to the colored polymeric material provided in (a);
(c) depolymerisation or transesterification of the polymeric material, which is depleted in colorant obtained in (b); thereby obtaining monomers and/or oligomers or transesterification products.

The inventive process represents a very simple and efficient method to discolor a polyalkylene terephthalate based polymer without affecting the polymeric structure, especially without degradation of the polyalkylene terephthalate based polymer. Thereafter the material can easily be chemically processed (via depolymerization or transesterification) without the need of extensive purification of the resulting products due to color.

“Contacting under conditions allowing for colorant removal but not allowing for polyester dissolution” in step (b) comprises any kind of contact between the colored polymeric material and the solvent system, as long as the colorant and optionally third polymer (iv) if present, are depleted, while the polyalkylene terephthalate based polymer is maintained non-degraded. As described in more detail below, this comprises especially in some embodiments that the colored polymeric material is contacted with gaseous solvent system and/or re-condensed solvent system, while in some alternative embodiments, the colored polymeric material is at least partially immersed in the (liquid) solvent system.

“Depleted in colorant” means that at least 50 weight-%, preferably at least 60 weight-%, more preferably at least 70 weight-%, more preferably at least 80 weight-%, more preferably at least 90 weight-%, more preferably at least 95 weight-%, of the colorant and, —if present—also the same perceptual amount of third polymer (iv) comprised in the colored polymeric material provided in (a), more preferably of the colorant comprised in the polyalkylene terephthalate based polymer and—if present—of the third polymer (iv) comprised in the colored polymeric material provided in (a), are removed from the polymeric material. “Enriched in colorant” means that at least 50 weight-%, preferably at least 60 weight-%, more preferably at least 70 weight-%, more preferably at least 80 weight-%, more preferably at least 90 weight-%, more preferably at least 95 weight-%, of the colorant and—if present—also the same perceptual amount of third polymer (iv) comprised in the material provided in (a) are dissolved in the solvent system.

### Colorant Removal in (b) Via Gaseous Stream

In some embodiments of the process, (b) comprises
(b.1) providing the colored polymeric material in a first vessel and providing the solvent system in a second vessel;
   wherein first and second vessel are arranged spatially separated from each other but allowing a gaseous and fluid communication between each other;
(b.2) forming a gaseous stream comprising at least parts of the solvent system, and removing the gaseous stream from the second vessel;
(b.3) allowing the solvent system to separate in liquid form from the gaseous stream at a position outside the second vessel (also outside the first vessel), obtaining a solvent system in liquid form;
(b.4) contacting the colored polymeric material in the first vessel with the solvent system in liquid form, which had been separated from the gaseous stream in (b.3),
   thereby obtaining a solvent system, which is enriched in colorant and optionally in third polymer (iv) compared to the solvent system provided in (a), and a polymeric material, which is depleted in colorant and optionally in third polymer (iv) compared to the colored polymeric material provided in (a) and which comprises the polyalkylene terephthalate based polymer (i) and optionally the second polymer (iii).

The expression “allowing a gaseous and fluid communication between each other” means that the colored polymeric material in the first vessel and the solvent system in the second vessel are not in direct contact with each other but that gases, gaseous phases and liquids can freely move between and around them.

The expression “a gaseous stream comprising at least parts of the solvent system” is to be understood in that first, a part of the solvent system remains in the second vessel and second, in that the gaseous stream comprises at least a part of the solvent system, wherein the solvent system is present in the gaseous stream in liquid state or gaseous state or partly in liquid and partly in gaseous state.

A position outside the second vessel, which is also outside the first vessel, is any position outside these two vessels, wherein the only requirement is that the solvent system can separate in liquid form from the gaseous stream and a transfer back to the polymeric material o in the first vessel by any suitable means is possible.

“Contacting” in these embodiments comprises especially that the colored polymeric material is contacted with gaseous solvent system and/or re-condensed solvent system and/or separated solvent system as described in more detail in the following.

In some embodiments of the process, (b.2) comprises
(b.2.1) heating the solvent system to a temperature at least equal to or above the boiling temperature of the solvent system;
(b.2.2) obtaining a gaseous stream comprising at least parts of the solvent system, and removing the gaseous stream from the second vessel.

In some embodiments, the solvent system, due to the heating thereof, at least parts thereof, are transferred from liquid into gaseous state. A “boiling point” of a solvent system is, if two or more solvents are present in the solvent system, understood as the boiling range of the solvent system in case of a zeotropic mixture, or, in case of an azeotropic mixture, the boiling point of said azeotropic mixture. The boiling point of a solvent is generally understood as the boiling point of the solvent at the respective pressure and the boiling point of a solvent system is equally understood as the boiling point of the solvent system at the respective pressure. In some embodiments of the process, additionally an inert gas is applied, which forms parts of the gaseous stream comprising at least parts of the solvent system.

In some embodiments of the process, (b.2) comprises
(b.2.1′) heating the solvent system to a temperature T of at most 3K below the boiling temperature of the solvent system; so that a solvent system having elevated temperature is obtained;
(b.2.2′) contacting the solvent system having elevated temperature with an inert gas, preferably passing inert gas through the solvent system having elevated temperature, so that a gaseous stream comprising at least parts of the solvent system is formed, and removing the gaseous stream from the second vessel.

Heating the solvent system in (b.2.1′) is done to a temperature in the range of 30° C., preferably 160° C., to at most 3K below the boiling temperature of the solvent system, wherein the 30 C or 160° C. preferably apply when step (b.ii.1′) is carried out at a pressure in the range of from 990 to 1030 hPa. In some embodiments of the process, at least one of (b.2), (b.3) and (b.4), preferably (b.1), (b.2), (b.3) and (b.4), is/are done at a pressure in the range of from 800 to 200,000 hPa; the same applies for all substeps described above and in the following. In some alternatively embodiments, at least one of at least one of (b.2), (b.3) and (b.4), preferably (b.1), (b.2), (b.3) and (b.4), is/are done at a pressure in the range of from 0.1 to 990 hPa, preferably in the range of from 1 to 990 hPa; the same applies for all substeps described above and in the following.

In some embodiments of the process, (b.3) comprises
(b.3.1) transferring the gaseous stream from the second vessel to a position outside the second vessel (also outside the first vessel);
(b.3.2) allowing the parts of the solvent system comprised in the gaseous stream to separate in liquid form from the gaseous stream at the position outside the second vessel (also outside the first vessel), obtaining a solvent system in liquid form.

Separation of the solvent system in (b.3.2) is done by any means suitable for separating a solvent system from a gaseous stream so that the solvent system is obtained in liquid form. For example, said separation is done by cooling the gaseous stream comprising at least parts of the solvent system, for example, by contacting with a surface of a means having a temperature lower than the temperature of the gaseous stream. In some embodiments, where the process is carried out in an extraction apparatus, such as a soxhlet extractor, the means having a temperature lower than the temperature of the solvent system is a condenser, preferably a condenser through which a cooling medium is conducted. Separation means, in case that the at least parts of the solvent system are at least partially in gaseous stream, condensation. In case that the at least parts of the solvent system are at least partially still in liquid state in gaseous stream, separation means detachment of these liquid parts from the gaseous stream.

In some embodiments of the process, (b.4) comprises
(b.4.1) transferring the solvent system in liquid form obtained in (b.3.2) to the colored polymeric material in the first vessel;
(b.4.2) contacting the colored polymeric material in the first vessel with the solvent system in liquid form,
   thereby obtaining a solvent system, which is enriched in colorant and optionally in third polymer compared to the solvent system provided in (a), and a polymeric material, which is depleted in colorant and optionally in third polymer compared to the colored polymeric material provided in (a) and which comprises the polyalkylene terephthalate based polymer and optionally the second polymer.

In some embodiments of the process, (b.4) comprises
(b.4.3) transferring the solvent system obtained in (b.4.2) into the second vessel, which contains the solvent system provided in (a).

In some embodiments of the process, first vessel containing the polymeric material and second vessel containing the solvent system are arranged spatially separated from each other in that the first vessel containing the polymeric material is arranged above the second vessel containing the solvent system. The expression “arranged above the second vessel containing the solvent” means that the polymeric material is arranged at a position which is vertically higher located than the position of the upper edge of the solvent system in the second vessel. Preferably, there is no direct material contact between polymeric material and solvent system, i.e. the polymeric material neither touches the surface of the solvent system nor is the polymeric material to any percentage thereof immersed in the solvent system in the vessel. Apparatuses usable for carrying out the method are not limited. For example, in some embodiments an apparatus comparable to a soxhlet extractor is used, wherein the second vessel comprising the solvent system is comparable to a still pot and the first vessel comprising the polymeric material is comparable to the extraction sleeve, the first vessel in some embodiments being a device having one or more opening(s) such as a sieve. A sieve can have any conceivable shape such as being a planar sieve, a curved sieve etc., as long as the sieve is capable of holding the polymeric material (in solid state) but allowing passage of liquids and gases through its openings.

In some embodiments of the process, (b.4.2) comprises contacting the colored polymeric material with the solvent system in liquid form of (b.4.1) in that the solvent system in liquid form drops onto the colored polymeric material.

In some embodiments of the process, (b.4.3) comprises transferring the solvent system, which is enriched in colorant and optionally in third polymer compared to the solvent system provided in (a), from the first to the second vessel in that the solvent system in liquid form drops from the first vessel (back) into the second vessel.

In some embodiments, the process comprises:
(b.1) providing the colored polymeric material in a first vessel and providing a solvent system in a second vessel;
   wherein first and second vessel are arranged spatially separated from each other in that the first vessel containing the colored polymeric material is arranged above the second vessel containing the solvent system and allowing a gaseous and fluid communication between each other (between first and second vessel);
(b.2) forming a gaseous stream comprising at least parts of the solvent system, and removing the gaseous stream from the second vessel;
(b.3.1) transferring the gaseous stream from the second vessel to a position outside the second vessel (also outside the first vessel);
(b.3.2) allowing the parts of the solvent system comprised in the gaseous stream to separate in liquid form from the gaseous stream at the position outside the second vessel (also outside the first vessel), preferably at a position above the first vessel, wherein said separation is preferably done by cooling the gaseous stream comprising at least parts of the solvent system, more preferably by contacting the gaseous stream at the position above the first vessel with a surface of a means having a temperature lower than the temperature of the gaseous stream; thereby obtaining a solvent system in liquid form;
(b.4.1) transferring the solvent system in liquid form obtained in (b.3.2) to the colored polymeric material in the first vessel in that the solvent system in liquid form drops from the position above the first vessel onto the colored polymeric material, which is in the first vessel;
(b.4.2) contacting the colored polymeric material in the first vessel with the solvent system in liquid form,
   thereby obtaining a solvent system, which is enriched in colorant and optionally in third polymer compared to the solvent system provided in (a), and a polymeric material, which is depleted in colorant and optionally in third polymer compared to the colored polymeric material provided in (a) and which comprises the polyalkylene terephthalate based polymer and optionally the second polymer; and preferably
(b.4.3) transferring the solvent system obtained in (b.4.2) into the second vessel, which contains the solvent system provided in (a), preferably in that the solvent system, which is enriched in colorant and optionally in third polymer compared to the solvent system provided in (a), in liquid form drops from the first vessel (back) into the second vessel.

For these embodiments, all preferred options described above with respect to (b.2.1), (b.2.1′), (b.2.2), and/or (b.2.2′) apply as well.

In some embodiments of the process, the inert gas is selected from the group consisting of argon, helium, neon, nitrogen and mixtures of two or more of these inert gases, preferably comprises at least nitrogen, more preferably the inert gas is nitrogen.

In some embodiments of the process, the inert gas is conducted through the solvent system and has a flow rate in the range of from 1 to 150 liter/h or is passed along the surface of the solvent system and has a flow rate of in the range of from 1 to 150 liter/h.

In some embodiments, the process further comprises
(b.5) removal of the solvent system obtained in (b.4) or (b.4.2), which is enriched in colorant and optionally in third polymer from the polymeric material (i.e. also out of the first vessel) and recirculation into the second vessel comprising the solvent system, preferably by dropping it into the second vessel comprising the solvent system (allowing the solvent system obtained in (b.4) or (b.4.2) or to drop into the second vessel comprising the solvent system).

In some embodiments of the process, (b.2), (b.3) and (b.4), preferably (b.2), (b.3), (b.4) and (b.5), are carried out in continuous mode.

In some embodiments the process further comprises
(b.6) at least partial removal of polymeric material obtained in (b.4) or (b.4.2), thereby obtaining a polymeric material which is depleted in colorant and optionally in third polymer (iv) compared to the colored polymeric material provided in (a), and which comprises the polyalkylene terephthalate based polymer and optionally the second polymer.

In some embodiments the process further comprises
(b.7) at least partial replacement of the polymeric material removed in (b.6) by fresh colored polymeric material without replacement of the solvent system provided in (a), wherein the fresh colored polymeric material comprises polyalkylene terephthalate based polymer, colorant, optionally second polymer and optionally third polymer, and repetition of step(s) (b.2), (b.3) and (b.4), optionally (b.5);
wherein steps (b.6) and (b.7) are preferably repeated at least one further time, preferably in the range of one to at least 10 further time(s) (batch mode); or wherein steps (b.6) and (b.7) are carried out continuously (purge mode).

In some embodiments of the process, in the range of from 1 to 50 weight-% of additional fresh solvent system, based on the total weight of the solvent system provided in (a) being 100 weight-% are added, wherein the addition is carried out discontinuously (batch mode) or continuously (purge mode).

### Colorant Removal in (b) by Treatment in Solution

In some alternatively embodiments of the process, (b.1) comprises
(b.1′) contacting the colored polymeric material of (a) with a solvent system at a temperature T, which is <170° C., thereby obtaining a solvent system, which is enriched in colorant and optionally in third polymer (iv) compared to the solvent system provided in (a), and a polymeric material, which is depleted in colorant and optionally in third polymer (iv) compared to the colored polymeric material provided in (a);
(b.2′) optionally separating the polymeric material, which is depleted in colorant and optionally in third polymer (iv), and which comprises the polyalkylene terephthalate based polymer, from the solvent system.

The separation in (b.2′) is done by methods and means known to the skilled person, especially solid-liquid separation methods such as filtration, for example, heated pressure filtration, sedimentation or centrifugation (see Handbuch der mechanischen Fest-Flüssig-Trennung Taschenbuch—29 Apr. 2004 von Klaus Luckert (Herausgeber)). Colorants remain at least partially in the separated solvent system obtained in (b.2′).

In these alternatively embodiments of the process, it is preferred that contacting in (b.1′) is done at a temperature T, which is in the range of from 10 to <170° C., preferably in the range of from 100 to <170° C., more preferably in the range of from 110 to <170° C., more preferably in the range of from 110 to 165° C., more preferably in the range of from 120 to 150° C.

“Contacting” in step (b.1′) preferably means that the colored polymeric material is at least partially immersed in the solvent system. Preferably, the colored polymeric material is at least partially immersed in the (first or second) solvent system in that at least 60%, more preferably at least 70%, more preferably at least 80%, more preferably at least 90%, more preferably at least 95%, more preferably at least 99% of the colored polymeric material's surface or are in contact with the solvent system, based on the total surface being 100%.

In these alternatively embodiments of the process, it is preferred that (b.1′) is done at a pressure in the range of from 800 to 200,000 hPa.

In some embodiments of the process, at least (b) is done in counter current mode. For example, if the contacting of step (b) is done within a vessel, the solvent system enters the vessel from one direction (either side or top/bottom) and the colored polymeric material enters the vessel from an another, preferably an opposite, direction. In a preferred constellation wherein a vertically arranged vessel is used, the solvent system enters the vessel from the bottom and the colored polymeric material enters the vessel from the top. In some embodiments of the process, at least (b) is conducted under mechanical intermixing, wherein mechanical intermixing preferably comprises one or more methods selected from stirring, blending, and ultra sound.

### Additional Steps

### Washing

In some embodiments of the process, ((b) comprises
(b.y) optionally washing the polymeric material separated in (b.6) or in (b.2′) with a washing solvent, thereby obtaining a washed polymeric material, which is depleted in colorant compared to the polymeric material provided in (a).

### Drying

In some embodiments of the process, (b) comprises
(b.z) drying the polymeric material separated in (b.6) or in (b.2′) and/or the washed polymeric material respectively obtained in (b.y).

Washing in optional step (b.y) is preferably done with a solvent system having features (s.1) or (s.1a), preferably (s.1) or (s.1a) and (s.2), more preferably (s.1) or (s.1a), (s.2) and (s.3), preferably with a solvent system comprising one or more of the solvent(s) of any one of the groups listed herein. Preferably, washing in optional step (b.y) is done with the same solvent system as used for color depletion. In some embodiments, washing is done with a solvent selected from the group consisting of methanol, ethanol, propanol, isopropanol, acetonitrile, ethyl acetate, acetone, water or a mixture of two or more of these solvents. In some embodiments, the washing in optional step (b.y) is done with a solvent selected from the group consisting of methanol, ethanol, propanol, isopropanol, acetonitrile, ethyl acetate, acetone, water, GVL, or a mixture of two or more of these solvents. Drying in step (b.z) is preferably done under one or more conditions selected from the group consisting of a pressure in the range of from 1 to 1013 mbar; a temperature in the range of from 50 to 210° C., preferably in the range of from 60 to 180° C., more preferably in the range of from 80 to 160° C.; drying time in the range of from 30 minutes to 24 hours; drying in an atmosphere comprising nitrogen, preferably in an atmosphere having at least 90 volume-%, more preferably 95 volume-%, more preferably at least 98 volume-% nitrogen. Drying is done by one or more methods selected from the group consisting of contact drying, convection drying and radiation drying.

### Solvent System

In some embodiments of the process, the solvent system comprises one or more solvent(s), wherein the solvent system
(s.1) has Hansen solubility parameters with respect to
   energy from dispersion forces between molecules (δDₛₛ),
   energy from dipolar intermolecular force between molecules (δPₛₛ) and
   energy from hydrogen bonds between molecules (δHₛₛ),
which fulfill equitation 1

(11)²≥4(δDₛₛ−17.5)²+(δPₛₛ−7.5)²+(δHₛₛ−7.5)² [equitation 1].

Preferably, the solvent system
(s.1a) has Hansen solubility parameters with respect to
   energy from dispersion forces between molecules (δDₛₛ),
   energy from dipolar intermolecular force between molecules (δPₛₛ) and
   energy from hydrogen bonds between molecules (δHₛₛ), which fulfill equitation 2

(8.8)²≥4(δDₛₛ−20)²+(δPₛₛ−11.8)²+(δHₛₛ−4.5)² [equitation 2].

Regarding the solvent system, any solvent system for which 4(δDₛₛ−17.5)²+(δPₛₛ−7.5)²+(δHₛₛ7.5)² is larger than (11) 2 (i.e. 121), preferably any solvent system for which 4 (δDₛₛ-20)²+(δPₛₛ11.8)²+(δHₛₛ−4.5)² is larger than (8.8) 2 (i.e. 77.44) is not suited to dissolve the colorant, while leaving the polyalkylene terephthalate based polymer almost completely undissolved non-degraded, and any solvent system for which 4(δDₛₛ−17.5)²+(δPₛₛ−7.5)²+(δHₛₛ−7.5)² is smaller or equal to (11) 2 (i.e. 121), preferably any solvent system for which 4(δDₛₛ−20)²+(δPₛₛ−11.8)²+(δHₛₛ−4.5)² is smaller or equal to (8.8)² (i.e. 77.44), is suitable for dissolving the colorant while leaving the polyalkylene terephthalate based polymer almost completely undissolved and nondegraded in the specific temperature range. In case of two or more solvents being part of the solvent system, i.e. n solvents with n being an integer with n≥2 and i=1 . . . n, the Hansen solubility parameters of the resulting mixture with respect to each of δDₛₛ, δHₛₛ and δPₛₛ are calculated, knowing the percentage part of each solvent in the solvent system, as the weighted arithmetic mean from δDₛᵢ, δHₛᵢ and δPₛᵢ of each of the n solvents S(i). The Hansen parameters are to be found in BIOVIA COSMOquick 2022.

Considering the three-dimensional form given by, for example, equitation 2 in the three-dimensional Hansen space, a sphere is formed which has its center at δD_{c}=20, δP_{c}=11.8 and δH_{c}=4.5 and a radius r of 8.8. The doubling of the dispersion parameter value is required, according to Charles Hansen, for achieving a spherical form. A Hansen sphere, since there are no negative values possible for δH, can also be considered as a dome, i.e. a half-sphere. The same principles apply for the three-dimensional form given by equitation 1 in the three-dimensional Hansen space.

In some embodiments of the process
(s.2) each solvent of the solvent system has a boiling point at 1013 hPa of at least 150° C., preferably of at least 160° C.

In some embodiments of the process
(s.3) solvents having a functional group selected from the group consisting of hydroxyl (OH), amino (NH₂), carboxyl (COOH), and thiol (SH) are excluded.

In some embodiments of the process
(s.3a) solvents having a functional group selected from the group consisting of hydroxyl (OH), amino (NH₂), secondary amine (—NH—), carboxyl (COOH), and thiol (SH) are excluded.

In some embodiments of the process, at least 90 weight-%, preferably at least 95 weight-%, more preferably at least 98 weight-%, more preferably in the range of from 99 to 100 weight-%, of the solvent system consist of two or more solvent(s), based on the total weight of the solvent system being 100 weight-%.

In some embodiments of the process, at least 90 weight-%, preferably at least 95 weight-%, more preferably at least 98 weight-%, more preferably in the range of from 99 to 100 weight-%, of the solvent system consist of one solvent, which fulfills equitation 1 and/or 2, based on the total weight of the solvent system being 100 weight-%.

In some embodiments of the process, the one or more solvent(s) of the solvent system is/are selected from the group consisting of N,N-dimethylbenzamide, N,N-dimethylphenylacetamide, 1,4-benzoquinone, acetophenone, dimethyl terephthalate, 1,3,5-trimethoxybenzene, 2-phenylacetophenone, N-methylcaprolactam, methylbenzoate, methyl-4-methoxybenzoate, butylene carbonate, propylene-glycol-dibenzoate, N-ethylpyrrolidone, benzophenone, di-benzyl malonate, N-ethyl-caprolactam, methyl 2-(5-oxotetrahydrofuran-3-yl)acetate (FAME), propiophenone, N-methoxypropyl-pyrrolidone, 1,4-cyclohexanedione, cyclohexane-carbonate, N-methoxyethyl-pyrrolidone, N, N-diethylphenylacetamide, phenyl acetate, 1-(2-hydroxyethyl) pyrrolidin-2-one acetate (HEPAc), N,N-diethylbenzamide, isopropyl-benzoate, cyclohexyl phenyl ketone, phenylacetic acid ethylester, phenylacetat, N-methyl-morpholine, benzyl-propionate, benzylacetate, Neopentyl-glycol-dibenzoate, tetrahydrofurfuryl acetate, N-methyl-imidazole, benzyl butyrate, 2-pyrrolidone, 2-phenoxyethanol propionate, 2-phenoxyethyl isobutyrate, N, N-dipropylbenzamide, N,N-dimethylacetamide, N,N-diethylacetamide, dihydrolevoglucosenon (Cyrene), propylenecarbonate, caprolactone, dimethylisosorbide, N-butylpyrrolidone, t-butylpyrrolidone, methyl-1-methyl-5-oxopyrrolidine-3-carboxylate (MMOC), gamma-valerolactone (GVL), delta-valerolactone, gamma butyrolactone, methyl 5-(dimethylamino)-2-methyl-5-oxopentanoate (Rhodiasolv Polarclean), caprolactam, phen ethyl acetate, methyl phenylacetate, benzyl benzoate, N, N-dimethyllactamide (Agnique AMD 3L), and dimethyl sulfoxide (DMSO).

In some embodiments of the process, the one or more solvent(s) of the solvent system is/are selected from the group consisting of dihydrolevoglucosenon (Cyrene), propylenecarbonate, caprolactone, dimethylisosorbide, N-butylpyrrolidone, t-butylpyrrolidone, methyl-1-methyl-5-oxopyrrolidine-3-carboxylate (MMOC), gamma-valerolactone (GVL), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, propyl benzoate, and dimethyl sulfoxide (DMSO).

In some embodiments of the process, the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, propyl benzoate and GVL.

In some embodiments of the process, the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, and propyl benzoate.

In some embodiments of the process, the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), methyl 5-(dimethylamino)-2-methyl-5-oxopentanoate (Rhodiasolv®Polarclean), phenethyl acetate, and GVL.

In some embodiments of the process, the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), methyl 5-(dimethylamino)-2-methyl-5-oxopentanoate (Rhodiasolv®Polarclean), and phenethyl acetate.

In some embodiments of the process, the one or more solvent(s) is/are selected from the group consisting of gamma valerolactone (GVL), N-butylpyrrolidone (NBP), propylenecarbonate, acetophenone, dimethyl sulfoxide (DMSO), dihydrolevoglucosenon (Cyrene), cyclohexanone and mixtures of two or more thereof. In some embodiments of the process, the one or more solvent(s) is/are selected from the group consisting of gamma valerolactone (GVL), N-butylpyrrolidone (NBP), propylenecarbonate, acetophenone, dimethyl sulfoxide (DMSO), dihydrolevoglucosenon (Cyrene) and mixtures of two or more thereof. In some embodiments of the process, the one or more solvent(s) is/are selected from the group consisting of gamma valerolactone (GVL), N-butylpyrrolidone (NBP), propylenecarbonate, acetophenone, dimethyl sulfoxide (DMSO) and mixtures of two or more thereof. In some embodiments of the process, the one or more solvent(s) is/are selected from the group consisting of gamma valerolactone (GVL), N-butylpyrrolidone (NBP), propylenecarbonate, acetophenone and mixtures of two or more thereof. In some embodiments of the process, the one or more solvent(s) is/are selected from the group consisting of gamma valerolactone (GVL), N-butylpyrrolidone (NBP), acetophenone and mixtures of two or more thereof. In some embodiments of the process, the one or more solvent(s) is/are selected from the group consisting of gamma valerolactone (GVL), N-butylpyrrolidone (NBP) and mixtures of GVL and NBP.

In some embodiments of the process, the one or more solvent(s) is/are selected from the group consisting of N-butylpyrrolidone (NBP), propylenecarbonate, acetophenone, dimethyl sulfoxide (DMSO), dihydrolevoglucosenon (Cyrene), cyclohexanone and mixtures of two or more thereof. In some embodiments of the process, the one or more solvent(s) is/are selected from the group consisting of N-butylpyrrolidone (NBP), propylenecarbonate, acetophenone, dimethyl sulfoxide (DMSO), dihydrolevoglucosenon (Cyrene) and mixtures of two or more thereof. In some embodiments of the process, the one or more solvent(s) is/are selected from the group consisting of N-butylpyrrolidone (NBP), propylenecarbonate, acetophenone, dimethyl sulfoxide (DMSO) and mixtures of two or more thereof. In some embodiments of the process, the one or more solvent(s) is/are selected from the group consisting of N-butylpyrrolidone (NBP), propylenecarbonate, acetophenone and mixtures of two or more thereof. In some embodiments of the process, the one or more solvent(s) is/are selected from the group consisting of N-butylpyrrolidone (NBP), acetophenone and mixtures of N-butylpyrrolidone (NBP) and acetophenone.

In some embodiments of the process, the one or more solvent(s) comprise at least N-butylpyrrolidone, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are N-butylpyrrolidone, based on a total weight of the one or more solvent(s) being 100 weight-%, more preferably the one solvent is N-butylpyrrolidone. In some embodiments, contacting in (b.1′) is done at a temperature T, which may be in the range of from 10 to <170° C., wherein T preferably may be in the range of from 140 to 160° C., more preferably in the range of from 150 to 160° C., or contacting in (b.1′) is done at a temperature T, which may be in the range of from 101 to 81 K below the boiling point of the of the N-butylpyrrolidone, preferably in the range of from 91 to 81 K below the boiling point of the of the N-butylpyrrolidone. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of N-butylpyrrolidone at the temperature T at which the process is run. The skilled person can determine and/or adjust said autogenous pressure according to the steam pressure curve of N-butylpyrrolidone at a certain temperature T. Steam pressure curves of N-butylpyrrolidone are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, the one or more solvent(s) comprise at least propylenecarbonate, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are propylenecarbonate, based on a total weight of the one or more solvent(s) being 100 weight-%, more preferably the one solvent is propylenecarbonate. In some embodiments, contacting in (b.1′) is done at a temperature T, which may be in the range of from 10 to <170° C., wherein T preferably may be in the range of from 140 to 160° C., more preferably in the range of from 150 to 160° C. or contacting in (b.1′) is done at a temperature T, which i may be in the range of from 102 to 82 K below the boiling point of the of the propylenecarbonate, preferably in the range of from 92 to 82 K below the boiling point of the of the propylenecarbonate. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of propylenecarbonate at the temperature T at which the process is run. The skilled person can determine and/or adjust said autogenous pressure according to the steam pressure curve of propylenecarbonate at a certain temperature T. Steam pressure curves of propylenecarbonate are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, the one or more solvent(s) comprise at least acetophenone, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are acetophenone, based on a total weight of the one or more solvent(s) being 100 weight-%, more preferably the one solvent is acetophenone. In some embodiments, contacting in (b.1′) is done at a temperature T, which may be in the range of from 10 to <170° C., wherein T preferably may be in the range of from 140 to 160° C., more preferably in the range of from 150 to 160° C. or contacting in (b.1′) is done at a temperature T, which may be in the range of from 62 to 42 K below the boiling point of the of the acetophenone, preferably in the range of from 52 to 42 K below the boiling point of the of the acetophenone. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of acetophenone at the temperature T at which the process is run. The skilled person can determine and/or adjust said autogenous pressure according to the steam pressure curve of acetophenone at a certain temperature T. Steam pressure curves of acetophenone are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, the one or more solvent(s) comprise at least dimethyl sulfoxide (DMSO), preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are DMSO, based on a total weight of the one or more solvent(s) being 100 weight-%, more preferably the one solvent is DMSO. In some embodiments, contacting in (b.1′) is done at a temperature T, which may be in the range of from 10 to <170° C., wherein T preferably may be in the range of from 140 to 160° C., more preferably in the range of from 150 to 160° C. or contacting in (b.1′) is done at a temperature T, which may be in the range of from 49 to 29 K below the boiling point of the of the DMSO, preferably in the range of from 39 to 29 K below the boiling point of the of the DMSO. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of DMSO at the temperature T at which the process is run. The skilled person can determine and/or adjust said autogenous pressure according to the steam pressure curve of DMSO at a certain temperature T. Steam pressure curves of DMSO are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, the one or more solvent(s) comprise at least dihydrolevoglucosenon (Cyrene), preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are Cyrene, based on a total weight of the one or more solvent(s) being 100 weight-%, more preferably the one solvent is Cyrene. In some embodiments, contacting in (b.1′) is done at a temperature T, which may be in the range of from 10 to <170° C., wherein T preferably may be in the range of from 140 to 160° C., more preferably in the range of from 150 to 160° C. or contacting in (b.1′) is done at a temperature T, which may be in the range of from 86 to 66 K below the boiling point of the of the Cyrene, preferably in the range of from 76 to 66 K below the boiling point of the of the Cyrene. The contacting in (b.1) or (b.2′) is preferably done at a pressure in the range of from 800 to 1200 hPa. Preferably, the process is run at autogenous pressure, wherein the autogenous pressure may be higher than the ambient pressure of the surrounding area caused by the steam pressure of Cyrene at the temperature T at which the process is run. The skilled person can determine and/or adjust said autogenous pressure according to the steam pressure curve of Cyrene at a certain temperature T. Steam pressure curves of Cyrene are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, the one or more solvent(s) comprise at least cyclohexanone, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are cyclohexanone, based on a total weight of the one or more solvent(s) being 100 weight-%, more preferably the one solvent is cyclohexanone. In some embodiments, contacting in (b.1′) is done at a temperature T, which may be in the range of from 10 to <170° C., wherein T preferably may be in the range of from 140 to 155° C., more preferably in the range of from 145 to 150° C. or contacting in (b.1′) is done at a temperature T, which may be in the range of from 16 to 1 K below the boiling point of the of the cyclohexanone, preferably in the range of from 11 to 6 K below the boiling point of the of the cyclohexanone. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of cyclohexanone at the temperature T at which the process is run. The skilled person can determine and/or adjust said autogenous pressure according to the steam pressure curve of cyclohexanone at a certain temperature T. Steam pressure curves of cyclohexanone are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

In some embodiments of the process, ethyl benzoate and butyl benzoate are excluded as solvent(s).

In some embodiments of the process, the solvent system comprises GVL, wherein preferably at least 90 weight-%, more preferably at least 95 weight-%, more preferably at least 98 weight-%, more preferably in the range of from 99 to 100 weight-%, based on the total weight of the solvent system being 100 weight-%, of the solvent system of the solvent system consist of GVL. n these embodiments, contacting in (b.1′) is done at a temperature T, which may be in the range of from 10 to <170° C., wherein T preferably may be in the range of from 140 to 160° C., more preferably in the range of from 150 to 160° C., or contacting in (b.1′) is done at a temperature T, which may be in the range of from 65 to 45 K below the boiling point of the of the GVL, preferably in the range of from 55 to 45 K below the boiling point of the of the GVL. The contacting in (b.1′) is preferably done at a pressure in the range of from 800 to 1200 hPa. Preferably, the process is run at autogenous pressure, wherein the autogenous pressure may be higher than the ambient pressure of the surrounding area caused by the steam pressure of GVL at the temperature T at which the process is run. The skilled person can determine and/or adjust said autogenous pressure according to the steam pressure curve of GVL at a certain temperature T. Steam pressure curves of GVL are known to a person skilled in the art. The autogenous pressure may be lowered by purging, e.g. by a purging valve, preferably to a pressure between ambient pressure and lower 2000 hPa, preferably between 1200 hPa and 1800 hPa. Autogenous pressure is the pressure caused by the production system itself in a closed system, e.g. a pressure in the range from 800 to 3000 hPa.

Preferably, the polyalkylene terephthalate based polymer of the polymeric material, which is depleted in colorant obtained in (b) has a number average molecular weight Mn, which is greater or at least equal to the Mn of the polyalkylene terephthalate based polymer comprised in the polymeric material provided in (a). Preferably, the polyalkylene terephthalate based polymer of the polymeric material, which is depleted in colorant obtained in (b) has a dispersity Mw/Mn (Mass average molecular weight Mw divided by number average molecular weight Mn) in the range of from 70 to 95%, preferably in the range of from 75 to 90% of the dispersity Mw/Mn of the polyalkylene terephthalate based polymer comprised in the polymeric material provided in (a) (100%).

### Depolymerisation in (c)

In some embodiments of the process, the depolymerisation of (c) is done by a method selected from the group consisting of alcoholysis, glycolysis, hydrolysis, aminolysis, ammonolysis, and mixtures of two or more of these methods.

In some embodiments of the process, the depolymerisation of (c) is done by alcoholysis, wherein (c) comprises contacting the polymeric material depleted in colorant obtained in (b) with a C₁ to C₄ alkyl mono alcohol, thereby obtaining a di (C₁ to C₄ alkyl) terephthalate and/or a di (C₁ to C₄ alkyl) isophthalate and ethylene glycol and/or cyclohexanedimethanol and/or butylene glycol.

In some embodiments of the process, the C₁ to C₄ alkyl mono alcohol is methanol, wherein di methyl terephthalate and/or di methyl isophthalate and ethylene glycol and/or cyclohexanedimethanol and/or butylene glycol are obtained.

In some embodiments of the process, the depolymerisation of (c) is done by glycolysis, wherein (c) comprises contacting the polymeric material depleted in colorant obtained in (b) with a C₁ to C₆ alkyl diol, thereby obtaining a bis(hydroxy C₁ to C₆ alkyl) terephthalate and/or bis(hydroxy C₁ to C₆ alkyl) isophthalate and ethylene glycol and/or cyclohexanedimethanol and/or butylene glycol.

In some embodiments of the process, the C₁ to C₆ alkyl diol is ethylene glycol, wherein bis(hydroxy ethylene) terephthalate and/or bis(hydroxy ethylene) isophthalate and and/or bis(hydroxy C₁ to C₆ alkyl) isophthalate and ethylene glycol and/or cyclohexanedimethanol and/or butylene glycol are obtained.

In some embodiments of the process, the depolymerisation of (c) is done by hydrolysis, wherein (c) comprises contacting the polymeric material depleted in colorant obtained in (b) with water, thereby obtaining terephthalic acid (TPA) and/or isophthalic acid and ethylene glycol and/or cyclohexanedimethanol and/or butylene glycol.

In some embodiments of the process, the depolymerisation of (c) is done by alkaline hydrolysis.

In some embodiments of the process, the hydrolysis is done by enzymatically catalyzed hydrolysis, wherein (c) comprises contacting the polymeric material depleted in colorant obtained in (b) with water in the presence of an enzyme, thereby obtaining terephthalic acid (TPA) and/or isophthalic acid and ethylene glycol and/or cyclohexanedimethanol and/or butylene glycol.

In some embodiments of the process, the depolymerisation of (c) is done by hydrolysis at a pressure in the range of from 30×10³ to 120×10³ hPa.

In some embodiments of the process, the depolymerisation of (c) is done by hydrolysis, wherein (c) comprises
(c.1) preparing a melt of the polymeric material, which is depleted in colorant, thereby obtaining a melt of the polymeric material, which is depleted in colorant;
(c.2) contacting the melt of the polymeric material obtained in (c.1) with a base, preferably an inorganic alkali metal based base, more preferably sodium hydroxide, potassium hydroxide or a mixture of sodium hydroxide and potassium hydroxide, thereby obtaining an alkali containing melt;
(c.3) contacting the alkali containing melt obtained in (c.2) with water, thereby obtaining terephthalate (with a corresponding alkali metal cation) and/or isophthalate (with a corresponding alkali metal cation) and ethylene glycol and/or cyclohexanedimethanol and/or butylene glycol.

In some embodiments of the process, the depolymerisation of (c) is done by aminolysis, wherein (c) comprises contacting the polymeric material depleted in colorant obtained in (b) with a mono alkyl amine R¹R²NH₂, wherein R¹ is C₁ to C₄ alkyl and R² is selected from hydrogen atom and C₁ to C₄ alkyl; thereby obtaining R¹R²NC(═O)-phenyl-C(═O)NR²R¹, wherein the substituents at the phenyl ring are in para or meta position, and ethylene glycol and/or cyclohexanedimethanol.

In some embodiments of the process, the depolymerisation of (c) is done by ammonolysis, wherein (c) comprises contacting the polymeric material depleted in colorant obtained in (b) with ammonia (NH₃), thereby obtaining H₂NC(═O)-phenyl-C(═O)NH₂, wherein the substituents at the phenyl ring are in para or meta position, and ethylene glycol and/or cyclohexanedimethanol; wherein the ammonia is provided solvent-free or dissolved in a solvent, wherein the solvent is preferably one or more of glycerin, glycol (preferably C₁ to C₆ alkyl diol), alcohol (preferably C₁ to C₄ alkyl mono alcohol).

### Transesterification of (c)

In some embodiments of the process, the transesterification of (c) is done by acid or base catalyzed reaction with an alcohol, wherein “acid” comprises Lewis acid and Brönsted acid and “base” comprises o Lewis base and Brönsted base, as known to the skilled person.

In some embodiments of the process, one or more catalysts containing metal cations are used, wherein the metal is preferably titanium or tin.

In some embodiments of the process, the alcohol is R³—OH, wherein R³ is a linear or branched C₂ to C₁₂ alkyl residue.

In some embodiments, the transesterification product is/are di (C₂ to C₁₂ alkyl) terephthalate and/or a di (C₂ to C₁₂ alkyl) isophthalate and ethylene glycol and/or cyclohexanedimethanol.

In some embodiments of the process, the alcohol is selected from the group consisting of 2-propyheptanol, 2-ethylhexanol, iso-propanol, isononanol, pentanol, butanol, isomers of these alcohols, and mixtures of two or more thereof.

Reaction conditions, catalysts etc. for the above-described reaction types are well known to the skilled person; reference is made, for example, to Raheem et al., Journal of Cleaner Production 225 (2019) 1052e1064, Paszun et al., Ind. Eng. Chem. Res. 1997, 36, 1373, and Barnard et al., Green Chem., 2021, 23, 3765-3789.

### Polyalkylene Terephthalate Based Polymer

The “polyalkylene terephthalate based polymer” consists of either oxyethylen units or oxybutylen units and oxyterephthaloyl units, wherein in case of oxyethylen units, in the range of from 0 to 5 mol-% of the oxyterephthaloyl units are replaced by oxyisophthaloyl units and/or in the range of from 0 to 49 mol-% of the oxyethylen units are replaced by oxymethylene cyclohexylene methylene units.

Preferably, the polyalkylene terephthalate based polymer is selected from the group consisting of PET (polyethylene terephthalate), PETG (poly(ethylene terephthalate-co-1,4-cyclohexylene dimethylene terephthalate)), PETI (poly(ethylene terephthalate-co-isophthalate)), PBT (polybutylene terephthalate), and mixtures of two or three of these polymers, or the polyalkylene terephthalate based polymer is selected from the group consisting of PET (polyethylene terephthalate), PETI (poly(ethylene terephthalate-co-isophthalate)), PBT (polybutylene terephthalate), and mixtures of two or three of these polymers.

In some embodiments, the polyalkylene terephthalate based polymer comprises at least 80 weight-%, more preferably at least 85 weight-%, more preferably at least 90 weight-%, more preferably at least 95 weight-%, more preferably at least 96 weight-%, more preferably at least 97 weight-%, of PET based on the total weight of the polyalkylene terephthalate based polymer being 100 weight-% and/or, preferably and, at the outmost 20 weight-%, more preferably at the outmost 15 weight-%, more preferably at the outmost 10 weight-%, more preferably at the outmost 5 weight-%, more preferably at the outmost 4 weight-%, more preferably at the outmost 3 weight-%, more preferably at the outmost 2 weight-%, more preferably at the outmost 1 weight-%, of PETI, based on the total weight of the polyalkylene terephthalate based polymer being 100 weight-%.

In some embodiments, the “polyalkylene terephthalate based polymer” comprises or is a polyester based on 1,4-butanediol or 1,2-ethandiol, more preferably a polyester selected from the group consisting of a polymer based on 1,4-butanediol and terephthalic acid (polybutylene terephthalate, PBT), a polymer based on 1,2-ethanediol and terephthalic acid (polyethylene terephthalate, PET), a copolymer of 1,4-butanediol, adipic acid and terephthalic acid (polybutylenadipat-terephthalat, PBAT), a polymer of 1,2-ethanediol and 2,5-furandicarboxylic acid (polyethylene furanoate, PEF) and mixtures of two or more of these (co) polymers.

More preferably, the “polyalkylene terephthalate based polymer” comprises at least PET and/or PBT, more preferably the polyester is PET or PBT or a mixture of PET and PBT.

More preferably the polyalkylene terephthalate based polymer comprises or is PET.

In some embodiments of the process, the colored polymeric material derives from a bottle and/or a textile. In some embodiments, the colored polymeric material derives from textiles such as clothing, wherein the textiles are preferably subjected to a sorting process before the polymeric material is subjected to a method according to the present invention. A sorting process preferably comprises one or more NIR sorting steps, wherein textiles are analysed by near-infrared (NIR) spectroscopy and, based on the analytic result, sorted based on their composition. Thus, the polymeric material subjected to the method according to the present invention is preferably a pre-sorted, more preferably an NIR presorted, textile. The textile has preferably underwent a size reduction, more preferably a cutting and/or shredding step. Thus, the polymeric material subjected to the method according to the present invention is preferably a pre-sorted, more preferably an NIR presorted, and/or size reduced, more preferably cutted and/or shredded, textile. In some embodiments, the polymeric material subjected to the method according to the present invention is preferably a pre-sorted, more preferably an NIR presorted, and/or size reduced, more preferably cutted and/or shredded, textile, which has a content of PA6 of less than 10 weight-%, more preferably of less than 5 weight-%, more preferably of less than 4 weight-%, more preferably of less than 3 weight-%, more preferably of less than 2 weight-%, more preferably of less than 1 weight-%, based on the total weight of the polymeric material being 100 weight-%. Lowering the content of PA in the textile may provide an improved quality of the obtained polyester, in particular of the polyalkylene terephthalate based polymer.

Second Polymer (iii)

In some embodiments of the process, the second polymer (iii) is selected from the group consisting of polypropylene (PP), polyethylene (PE), polyamide (PA), preferably PA6 and/or PA66, natural polymer, preferably cotton, viscose, linen and mixtures of two or more thereof.

### Third Polymer (iv)

In some embodiments of the process, the third polymer (iv) is an elastic fiber.

In some embodiments of the process, the elastic fiber comprises, preferably consists of one or more polyurethane based elastic fiber(s) and/or one or more polyester based elastic fiber(s), more preferably the elastic fiber comprises, preferably consists of one or more polyurethane based elastic fiber(s), wherein more preferably at least 40 weight-%, more preferably at least 45 weight-%, more preferably at least 50 weight-%, more preferably at least 55 weight-%, more preferably at least 60 weight-%, more preferably at least 65 weight-%, more preferably at least 70 weight-%, more preferably at least 75 weight-%, more preferably at least 80 weight-%, more preferably at least 85 weight-%, more preferably at least 90 weight-%, more preferably at least 95 weight-%, more preferably at least 99.9 weight-%, of the elastic fiber are polyurethane based elastic fiber(s), each based on the total weight of the elastic fiber being 100 weight-%.

The polyurethane based elastic fiber(s) is/are preferably (block) copolymers based on polyurethane. A (block) copolymer based on polyurethane is preferably a (block) copolymer of polyurethane and one or more polyether(s) selected from the group consisting of polyethylene glycol, polytetrahydrofurane, a copolymer of 2-methyl-tetrahydrofurane and tetrahydrofurane, and a copolymer of 3-methyl-tetrahydrofurane and tetrahydrofurane, wherein the (block) copolymer based on polyurethane is more preferably a (block) copolymer of polyurethane and polyethylene glycol or a (block) copolymer of polyurethane and polytetrahydrofurane, wherein more preferably in each of these (block) copolymers of polyurethane, the polyurethane content is at least 85 weight-%, based on the total weight of the (block) copolymer being 100 weight-%. The expression “(block) copolymer” means copolymer and blockcopolymer, wherein blockcopolymer is preferred. (Block) copolymers of polyurethane with one or more polyether(s) selected from the group consisting of polyethylene glycol, polytetrahydrofurane, a copolymer of 2-methyl-tetrahydrofurane and tetrahydrofurane, a copolymer of 3-methyl-tetrahydrofurane and tetrahydrofurane, especially when the polyurethane content is at least 85 weight-% based on the total weight of the (block) copolymer being 100 weight-%, are also called by generic names such as “spandex”, “elastan (e)”, “elastano”, “elastam”, “elastaan” or “lycra”. Brand names include also “Lycra”, “Elaspan”, “Acepora”, “Creora”, “INVIYA”, “ROICA”, “Dorlastan”, “Linel” and “ESPA”. A polyester based elastic fiber is preferably a poly(trimethylene terephthalate) (PTT) copolymer, wherein the poly(trimethylene terephthalate) copolymer is more preferably selected from the group of copolyesters synthesized from 2 or more reactants, each reactant having two functional groups capable of forming ester groups. For example, a poly(trimethylene terephthalate) copolymer may be prepared by reacting 1,3-propanediol and terephthalic acid, and optionally one or more comonomers selected from the group consisting of linear aliphatic dicarboxylic acids having 4 to 12 carbon atoms, cyclic aliphatic dicarboxylic acids having 4 to 12 carbon atoms, branched aliphatic dicarboxylic acids having 4 to 12 carbon atoms (such as butanedioic acid, pentanedioic acid, hexanedioic acid, azelaic acid, sebacic acid, dodecanedioic acid, 1,4-cyclohexanedicarboxylic acid, or ester-forming equivalents thereof), aromatic dicarboxylic acids other than terephthalic acid having 8 to 12 carbon atoms (such as phthalic acid, isophthalic acid or 2,6-naphthalenedicarboxylic acid); linear diols other than 1,3-propanediol having 2 to 8 carbon atoms, cyclic diols having 2 to 8 carbon atoms, and branched aliphatic diols having 2 to 8 carbon atoms (such as ethanediol, 1,2-propanediol, 1,4-butanediol, hexamethylene glycol, 3-methyl-1,5-pentanediol, 2,2-dimethyl-1,3-propanediol, 2-methyl-1,3-propanediol, cyclohexane dimethanol or 1,4-cyclohexanediol), aliphatic ether glycols having 4 to 10 carbon atoms and aromatic ether glycols having 4 to 10 carbon atoms (such as hydroquinone bis(2-hydroxyethyl) ether). Alternatively, a poly(trimethylene terephthalate) copolymer may be prepared from a poly(ethylene ether) glycol having a molecular weight below 460 g/mol, such as diethylene ether glycol, methoxypolyalkylene glycol, diethylene glycol, and polyethylene glycol. The comonomer is present in the copolymer in the range of from 0.5 to 30 mol %, preferably in the range of from 0.5 to 20 mol %. A generic name for a poly(trimethylene terephthalate) (PTT) copolymer is “Sorona”, wherein Sorona is in some embodiments a copolymer of 1,3-propane diol (preferably obtained by formation) and terephthalic acid (TPA) or dimethyl terephthalate (DMT), wherein preferably in the range of from 20 to 50 weight-%, more preferably in the range of from 30 to 40 weight-% of the copolymer are based on 1,3-propane diol, more preferably 1,3-propane diol obtained from renewable resources.

The colored polymeric material comprises (i) a polyalkylene terephthalate based polymer, (ii) a colorant and optionally (iii) a second polymer different from the polyalkylene terephthalate based polymer of (i) and optionally (iv) a third polymer different from the polyalkylene terephthalate based polymer of (i) and different from the second polymer of (iii), wherein the second polymer optionally is selected from the group consisting of poly propylene (PP), polyethylene (PE), polyamide (PA), preferably PA6 and/or PA66, natural polymer, preferably cotton, viscose, linen and mixtures of two or more thereof; and/or wherein the third polymer optionally is an elastic fiber, which preferably comprises, preferably consists of one or more polyurethane based elastic fiber(s) and/or one or more polyester based elastic fiber(s), more preferably the elastic fiber comprises, preferably consists of one or more polyurethane based elastic fiber(s), wherein more preferably at least 40 weight-%, more preferably at least 45 weight-%, more preferably at least 50 weight-%, more preferably at least 55 weight-%, more preferably at least 60 weight-%, more preferably at least 65 weight-%, more preferably at least 70 weight-%, more preferably at least 75 weight-%, more preferably at least 80 weight-%, more preferably at least 85 weight-%, more preferably at least 90 weight-%, more preferably at least 95 weight-%, more preferably at least 99.9 weight-%, of the elastic fiber are polyurethane based elastic fiber(s), each based on the total weight of the elastic fiber being 100 weight-%. Optionally, at least 90 weight-%, preferably at least 95 weight-%, more preferably at least 97 weight-% of the colored polymeric material consist of (i) polyalkylene terephthalate based polymer, (ii) colorant and optionally (iv) third polymer, based on the total weight of the colored polymeric material being 100 weight-%, wherein the third polymer optionally is an elastic fiber, which preferably comprises, preferably consists of one or more polyurethane based elastic fiber(s) and/or one or more polyester based elastic fiber(s), more preferably the elastic fiber comprises, preferably consists of one or more polyurethane based elastic fiber(s), wherein more preferably at least 40 weight-%, more preferably at least 45 weight-%, more preferably at least 50 weight-%, more preferably at least 55 weight-%, more preferably at least 60 weight-%, more preferably at least 65 weight-%, more preferably at least 70 weight-%, more preferably at least 75 weight-%, more preferably at least 80 weight-%, more preferably at least 85 weight-%, more preferably at least 90 weight-%, more preferably at least 95 weight-%, more preferably at least 99.9 weight-%, of the elastic fiber are polyurethane based elastic fiber(s), each based on the total weight of the elastic fiber being 100 weight-%. Optionally, less than 10 weight-%, preferably less than 5 weight-%, more preferably less than 3 weight-% of the colored polymeric material are (iii) second polymer, based on the total weight of the colored polymeric material being 100 weight-%, wherein the second polymer optionally is selected from the group consisting of poly propylene (PP), polyethylene (PE), polyamide (PA), preferably PA6 and/or PA66, natural polymer, preferably cotton, viscose, linen and mixtures of two or more thereof. Optionally, at least 90 weight-%, preferably at least 95 weight-%, more preferably at least 97 weight-% of the colored polymeric material consist of (i) polyalkylene terephthalate based polymer, (ii) colorant and optionally (iii) second polymer, based on the total weight of the colored polymeric material being 100 weight-%, wherein the second polymer optionally is selected from the group consisting of poly propylene (PP), polyethylene (PE), polyamide (PA), preferably PA6 and/or PA66, natural polymer, preferably cotton, viscose, linen and mixtures of two or more thereof. Optionally, less than 10 weight-%, preferably less than 5 weight-%, more preferably less than 3 weight-% of the colored polymeric material are (iv) third polymer, based on the total weight of the colored polymeric material being 100 weight-%, wherein the third polymer optionally is an elastic fiber, which preferably comprises, preferably consists of one or more polyurethane based elastic fiber(s) and/or one or more polyester based elastic fiber(s), more preferably the elastic fiber comprises, preferably consists of one or more polyurethane based elastic fiber(s), wherein more preferably at least 40 weight-%, more preferably at least 45 weight-%, more preferably at least 50 weight-%, more preferably at least 55 weight-%, more preferably at least 60 weight-%, more preferably at least 65 weight-%, more preferably at least 70 weight-%, more preferably at least 75 weight-%, more preferably at least 80 weight-%, more preferably at least 85 weight-%, more preferably at least 90 weight-%, more preferably at least 95 weight-%, more preferably at least 99.9 weight-%, of the elastic fiber are polyurethane based elastic fiber(s), each based on the total weight of the elastic fiber being 100 weight-%. Optionally, at least 90 weight-%, preferably at least 95 weight-%, more preferably at least 97 weight-% of the colored polymeric material consist of (i) polyalkylene terephthalate based polymer and (ii) colorant, based on the total weight of the colored polymeric material being 100 weight-%. In some embodiments, less than 10 weight-%, preferably less than 5 weight-%, more preferably less than 3 weight-% of the colored polymeric material are (iii) second polymer and (iv) third polymer, based on the total weight of the colored polymeric material being 100 weight-%.

### Colorant

In some embodiments of the process, the colorant is selected from the group consisting of dye and optical brightener and mixtures of dye and optical brightener.

A “colorant” is a substance that cause the change of color impression of material. This comprises dyes, which absorb wavelength intervals of visible light (400 to 780 nm) and optical brighteners, which amplify the light emission of a material through UV light adsorption and emittance of visible light (through fluorescence), i.e. an optical brightener converts radiation that is not visible to the human eye (<400 nm) into visible fluorescence radiation of the blue-red spectral range (400 to 600 nm). Colorants usable or used for changing the color impression of polymeric materials are known to the skilled person. In the context of the present invention, the term “dye” means any kind of dye such as dye, pigment, dispersion, wherein a dye is, for example, one or more selected from the group consisting of acid dye, basic dye, direct dye, disperse dye, azoic dye, food dye, solvent dye, organic dye, organic pigment, sulfur dye, mordant dye and vat dye. The term “optical brightener” comprises optical brightening agents, fluorescent brightening agents, and fluorescent whitening agents.

Overviews of colorants for polymeric materials can be found, for example, in “Dyes and Pigments” Metin Açikyildiz, Kübra Günes, Ahmet Gürses Springer, 2016 (ISBN: 10: 3319338900); Industrial Organic Pigments-Klaus Hunger, Thomas Heber, Martin U. Schmidt, Friedrich Reisinger, Stefan Wanne Wiley-VCH, 4^{th} edition, 2018 (ISBN: 978-3-527-32608-2); Chemistry and Technology of Natural and Synthetic Dyes and Pigments—Ashis Kumar Samanta, Nasser Awwad, IntechOpen, 2020 (ISBN: 9781789859980, 9781789859973, 9781839687587); Encyclopedia of Color, Dyes, Pigments—Volume 1, Gerhard Pfaff, de Gruyter, 2021 (ISBN: 311058588X); Heinrich Zollinger: Color Chemistry: Syntheses, Properties, and Applications of Organic Dyes and Pigments. 3^{rd} edition. WILEY-VCH Verlag, Weinheim 2003 (ISBN: 3-906390-23-3); Klaus Hunger (Ed.): Industrial Dyes: Chemistry, Properties, Applications. WILEY-VCH Verlag, Weinheim 2003 (ISBN: 3-662-01950-7); Hermann Rath: Lehrbuch der Textilchemie. ein-schl. der textilchemischen Technologie. 2nd edition. Springer-Verlag, Berlin, Heidelberg 1963 (ISBN: 978-3-662-00065-6); Wilfried Kratzert, Rasmus Peichert: Farbstoffe. Quelle & Meyer, Heidelberg 1981 (ISBN: 3-494-01021-8); Ullmann's Encyclopedia of industrial chemistry, WileyVCH, 2000, sections “dyes and pigments” and “dyes, general survey” (ISBN: 9783527303854). In some embodiments of the process, depleted in colorant regarding the polymeric material obtained means that the L*a*b* values of the polymeric material, which is depleted in colorant compared to the polymeric material provided in (a), change in that:
the absolute value of a* decreases, preferably by at least 0.2; and/or, preferably and,
the absolute value of b* changes, preferably by at least 0.2; and/or, preferably and,
the L* value increases, preferably by at least 4,
each compared to the L*a*b* values of the colored polymeric material provided in (a), wherein L*a*b* values are determined according to DIN 5033 and DIN EN ISO 11664-1.6.

The expression “irrespective of the color” means that, even if analytics are normally done for materials of each color separately, the definitions given above apply for single-colored polymeric materials, but also for polymeric materials having a plurality of colors and mixtures of pieces of polymeric materials, wherein each piece has its own color or its own color mix.

The condition of being “depleted in colorant”, which is expressed above based on quantitative L*a*b* values is also identifiable visually by the eye: The polymeric material provided in (a) has a certain color, wherein the polymeric material obtained, preferably obtained from (b), (b.4), (b.2.′) or (b6), as described herein is lighter and whiter respectively. This applies especially for all colorants not being optical brighteners. Depleted in colorant regarding the polymeric material obtained means, especially with respect to optical brighteners being the colorant, that the intensity of emitted fluorescence radiation (emission), preferably in the range of from 400 to 600 nm, is reduced for the polymeric material obtained in(s) when irradiated with light with a wavelength in the range of from 250 to 400 nm compared to the intensity of emitted fluorescence radiation (emission), preferably in the range of from 400-600 nm, of the polymeric material provided in (a).

Methods for determination of the intensity of emitted fluorescence radiation are known to the skilled person, for example, the determination can be made visually by using an UV lamp, by fluorescence determination or determination of quantum yield.

Separation of Second Polymer (iii)

In some embodiments the process further comprises, if second polymer (iii) was present in the polymeric material provided in (a):
(d) separating the second polymer (iii) from the monomers and/or oligomers or transesterification products obtained in (c), thereby obtaining separate monomers and/or oligomers or transesterification products and separate second polymer (iii).

### Separation of Third Polymer (iv)

In some embodiments the process further comprises, if third polymer (iv) was present in the polymeric material provided in (a):
(b.z) separating the third polymer (iv) from the solvent system, which is enriched in colorant obtained in (b), (b.4), (b.4.2.), (b.5) and/or (b.2′), thereby obtaining separate third polymer (iv).

Separation of the third polymer, if present in the polymeric material, and optionally of colorant is done by, for example, distillation, wherein the solvent system is removed and the remaining residue is put to a further use.

In some embodiments, the process comprises recycling separated solvent system obtained in one or more of the process steps described above back into the process, optionally after one or more work-up step(s).

### 2^{nd} Aspect—Monomer, Oligomer or Transesterification Product of a Polyalkylene Terephthalate Based Polymer

A second aspect of the invention is directed to a monomer, oligomer or transesterification product of a polyalkylene terephthalate based polymer obtained or obtainable by the process of the first aspect, preferably of step (d) All details and embodiments, as well as alternatively embodiments disclosed above in the section related to the first aspect apply also to the second aspect.

3^{rd} aspect—Re-polymerisation of monomers, oligomers and/or transesterification products

The invention relates in a fourth aspect to a process, preferably according to the first aspect, comprising the further step:
converting, preferably polymerizing, the monomers, oligomers or transesterification products of a polyalkylene terephthalate based polymer obtained or obtainable according to the process of the first aspect to obtain one or more polymer or polymer product.

All details and embodiments, as well as alternatively embodiments disclosed above in the section related to the first aspect apply also to the third aspect.

Preferably, the one or more polymer is/are polyalkylene terephthalate based polymer The “polyalkylene terephthalate based polymer” consists of either oxyethylen units or oxybutylen units and oxyterephthaloyl units, wherein in case of oxyethylen units, in the range of from 0 to 5 mol-% of the oxyterephthaloyl units are replaced by oxyisophthaloyl units and/or in the range of from 0 to 49 mol-% of the oxyethylen units are replaced by oxymethylene cyclohexylene methylene units. Preferably, the polyalkylene terephthalate based polymer is selected from the group consisting of PET (polyethylene terephthalate), PETG (poly(ethylene terephthalate-co-1,4-cyclohexylene dimethylene terephthalate)), PETI (poly(ethylene terephthalate-co-isophthalate)), PBT (polybutylene terephthalate), and mixtures of two or more of these polymers, or the polyalkylene terephthalate based polymer is selected from the group consisting of PET (polyethylene terephthalate), PETI (poly(ethylene terephthalate-co-isophthalate)), PBT (polybutylene terephthalate), and mixtures of two or three of these polymers.

In some embodiments, the “polyalkylene terephthalate based polymer” is a polyester based on 1,4-butanediol or 1,2-ethandiol, more preferably a polyester selected from the group consisting of a polymer based on 1,4-butanediol and terephthalic acid (polybutylene terephthalate, PBT), a polymer based on 1,2-ethanediol and terephthalic acid (polyethylene terephthalate, PET), a copolymer of 1,4-butanediol, adipic acid and terephthalic acid (polybutylenadipat-terephthalat, PBAT), a polymer of 1,2-ethanediol and 2,5-furandicarboxylic acid (polyethylene furanoate, PEF) and mixtures of two or more of these (co) polymers.

More preferably, the “polyalkylene terephthalate based polymer” comprises at least PET and/or PBT, more preferably the polyester is PET or PBT or a mixture of PET and PBT.

More preferably the polyalkylene terephthalate based polymer comprises or is PET.

Preferably, the polymer product is a textile, a fiber, a packaging, a plastic, preferably:
a part of a car; preferably cylinder head cover, engine cover, housing for charge air cooler, charge air cooler flap, intake pipe, intake manifold, connector, gear wheel, fan wheel, cooling water box, housing, housing part for heat exchanger, coolant cooler, charge air cooler, thermostat, water pump, radiator, fastening part, part of battery system for electromobility, dashboard, steering column switch, seat, headrest, center console, transmission component, door module, A, B, C or D pillar cover, spoiler, door handle, exterior mirror, windscreen wiper, windscreen wiper protection housing, decorative grill, cover strip, roof rail, window frame, sunroof frame, antenna panel, headlight and taillight, engine cover, cylinder head cover, intake manifold, airbag, cushion, or coating;
a cloth; preferably shirt, trousers, pullover, boot, shoe, shoe sole, tight or jacket;
an electrical part; preferably electrical or electronic passive or active component, circuit board, printed circuit board, housing component, foil, line, switch, plug, socket, distributor, relay, resistor, capacitor, inductor, bobbin, lamp, diode, LED, transistor, connector, regulator, integrated circuit (IC), processor, controller, memory, sensor, microswitch, microbutton, semiconductor, reflector housing for light-emitting diodes (LED), fastener for electrical or electronic component, spacer, bolt, strip, slide-in guide, screw, nut, film hinge, snap hook (snap-in), or spring tongue;
a consumer, agricultural product or pharmaceutical product; preferably tennis string, climbing rope, bristle, brush, artificial grass, 3D printing filament, grass trimmer, zipper, hook and loop fastener, paper machine clothing, extrusion coating, fishing line, fishing net, offshore line and rope, vial, syringe, ampoule, bottle, sliding element, spindle nut, chain conveyor, plain bearing, roller, wheel, gear, roller, ring gear, screw and spring dampers, hose, pipeline, cable sheathing, socket, switch, cable tie, fan wheel, carpet, box or bottle for cosmetics, mattress, cushion, insulation, detergent, dishwasher tabs or powder, shampoo, body wash, shower gel, soap, fertilizer, fungicide, or pesticide;
a packaging for the food industry; preferably mono- or multi-layer blown film, cast film (mono- or multi-layer), biaxially stretched film, or laminating film; or
a part of a construction; preferably a rotor blade, insulating material, frame, housing, wall, coating, or separating wall.

The converting, preferably polymerization steps to convert the monomers, oligomers or transesterification products of a polyalkylene terephthalate based polymer obtained for re-polymerisation to a polyalkylene terephthalate based polymer may comprise one or more synthesis steps and can be performed by conventional synthesis and technics well known to a person skilled in the art (see, for example, Ullmann's Encyclopedia of Industrial Chemistry, section “Polyesters”, Wiley-VCH Verlag Gmbh & Co. KGaA. 2018, 10.1002/14356007.a21_227.pub2).

4^{th} Aspect—Conversion of Second Polymer (iii) and/or Third Polymer (iv)

The invention relates in a fourth aspect to a process, preferably according to the first aspect, comprising the further step:
converting the separated second polymer (iii) and/or the separated third polymer (iv) obtainable by or obtained by the process of the first aspect, preferably the separated second polymer (iii) obtained or obtainable from step (d) and/or the separated third polymer (iv) obtained or obtainable from any one of steps (b), (b.4), (b.4.2.), (b.5) and (b.2′), to obtain one or more monomer, polymer or polymer product.

All details and embodiments, as well as alternatively embodiments disclosed above in the section related to the first aspect apply also to the fourth aspect.

Preferably, the polymer is and/or the polymer product comprises polyamide (PA); preferably PA 6 or PA 66; polyisocyanate polyaddition product; preferably polyurethane (PU), thermoplastic polyurethane (TPU), polyurea or polyisocyanurate (PIR); low-density polyethylene (LDPE), high-density polyethylene (HDPE), polyethylene (PE), polypropylene (PP), polyvinyl chloride (PVC), polyvinyl acetate (PVA), polystyrene (PS), poly acrylonitrile butadiene styrene (ABS), poly styrene acrylonitrile (SAN), poly acrylate styrene acrylonitrile (ASA), polytetrafluoroethylene (PTFE), poly(methyl acrylate) (PMA), poly(methyl methacrylate) (PMMA), polybutadiene (BR, PBD), poly(cis-1,4-isoprene), poly(trans-1,4-isoprene), polyoxymethylene (POM), polyethylene terephthalate (PET), polybutylene terephthalate (PBT), polybutylene adipate coterephthalate (PBAT), polyester (PES), polyether sulfone (PESU), polyhydroxyalkanoate (PHA), poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), polylactic acid (PLA), polysulfone (PSU), polyphenylene sulfone (PPSU), polycarbonate (PC), polyether ether ketone (PEEK), poly(p-phenylene oxide) (PPO), poly(p-phenylene ether) (PPE); or copolymer or mixture thereof.

Preferably, the polymer and/or the polymer product is/are or is/are a part of:
a part of a car; preferably cylinder head cover, engine cover, housing for charge air cooler, charge air cooler flap, intake pipe, intake manifold, connector, gear wheel, fan wheel, cooling water box, housing, housing part for heat exchanger, coolant cooler, charge air cooler, thermostat, water pump, radiator, fastening part, part of battery system for electromobility, dashboard, steering column switch, seat, headrest, center console, transmission component, door module, A, B, C or D pillar cover, spoiler, door handle, exterior mirror, windscreen wiper, windscreen wiper protection housing, decorative grill, cover strip, roof rail, window frame, sunroof frame, antenna panel, headlight and taillight, engine cover, cylinder head cover, intake manifold, airbag, cushion, or coating;
a cloth; preferably shirt, trousers, pullover, boot, shoe, shoe sole, tight or jacket;
an electrical part; preferably electrical or electronic passive or active component, circuit board, printed circuit board, housing component, foil, line, switch, plug, socket, distributor, relay, resistor, capacitor, inductor, bobbin, lamp, diode, LED, transistor, connector, regulator, integrated circuit (IC), processor, controller, memory, sensor, microswitch, micro-button, semiconductor, reflector housing for light-emitting diodes (LED), fastener for electrical or electronic component, spacer, bolt, strip, slide-in guide, screw, nut, film hinge, snap hook (snap-in), or spring tongue;
a consumer, agricultural product or pharmaceutical product; preferably tennis string, climbing rope, bristle, brush, artificial grass, 3D printing filament, grass trimmer, zipper, hook and loop fastener, paper machine clothing, extrusion coating, fishing line, fishing net, offshore line and rope, vial, syringe, ampoule, bottle, sliding element, spindle nut, chain conveyor, plain bearing, roller, wheel, gear, roller, ring gear, screw and spring dampers, hose, pipeline, cable sheathing, socket, switch, cable tie, fan wheel, carpet, box or bottle for cosmetics, mattress, cushion, insulation, detergent, dishwasher tabs or powder, shampoo, body wash, shower gel, soap, fertilizer, fungicide, or pesticide;
a packaging for the food industry; preferably mono- or multi-layer blown film, cast film (mono- or multi-layer), biaxially stretched film, or laminating film; or
a part of a construction; preferably a rotor blade, insulating material, frame, housing, wall, coating, or separating wall.

Preferably, the content of the separated second polymer (iii) and/or the separated third polymer (iv) in the polymer and/or polymer product is 1 weight-% or more, preferably 2 weight-% or more, more preferably 5 weight-% or more, more preferably 15 weight-% or more, more preferably 30 weight-% or more, more preferably 40 weight-% or more, more preferably 60 weight-% or more, more preferably 80 weight-% or more, more preferably 90 weight-% or more, more preferably 95 weight-% or more; and/or
the content of the separated second polymer in the polymer and/or polymer product is 100 weight-% or less, preferably 95 weight-% or less, more preferably 90 weight-% or less, more preferably 50 weight-% or less, more preferably 25 weight-% or less, more preferably 10 weight-% or less; and
wherein preferably the content is determined based on identity preservation and/or segregation and/or mass balance and/or book and claim chain of custody models, preferably based on mass balance, preferably the International Sustainability and Carbon Certification (ISCC) standard.

The converting steps to obtain the monomer, polymer or polymer product from the separated second polymer (iii) and/or the separated third polymer (iv) may comprise one or more synthesis steps and can be performed by conventional synthesis and technics well known to a person skilled in the art. Independent of the person skilled in the art to assess novelty and inventive step of the independent claim(s), the person skilled in the art to perform the converting step(s) is preferably from the technical field(s) pyrolysis, gasification, remonomerization, depolymerization, synthesis, production of monomers, polymers and polymer compounds, and/or its further processing (e.g. extrusion, injection molding). Examples of the step(s) of the conversion is/are described in “Industrial Organic Chemistry”, 3. volume, Wiley-VCH, 1997, ISBN: 978-3-527-28838-0, “Kunststoffhandbuch”, 11 volumes in 17 sub-volumes, Carl Hanser Verlag; especially volume 6, “Polyamide”, 1. edition, 1966, volume 7, “Polyurethane”, 3. edition, 1993, and volume 8, “Polyester”, 2. edition 1973; “Industrial Organic Chemistry”, 3. volume, Wiley-VCH, 1997, ISBN: 978-3-527-28838-0, “Injection Molding Reference Guide, 4th edition, CreateSpace Independent Publishing Platform, 2011, ISBN: 978-1466407824, EP 0989146 A1, EP 1460094 A1, WO 2006034800 A1, EP 1529792 A1, WO 2006042674 A1, EP 0364854 A2, U.S. Pat. No. 5,506,275 A, EP 0897402 A1, WO 2015082316 A1, WO 2021021855 A1, WO 2021126938 A1, WO 2021021902 A1, WO 2021092311 A1, WO 2008155271 A1, WO 2013139827 A1, each of which is incorporated herein by reference.

The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. A process for recycling of a colored polymeric material, wherein the colored polymeric material comprises (i) a polyalkylene terephthalate based polymer poly[CH₂]ₓ terephthalate, wherein x is an integer in the range of from 1 to 4, (ii) a colorant and optionally (iii) a second polymer different from the polyalkylene terephthalate based polymer of (i) and optionally (iv) a third polymer different from the polyalkylene terephthalate based polymer of (i) and different from the second polymer of (iii), the process comprising
   (a) providing a colored polymeric material and a solvent system;
   (b) contacting the colored polymeric material provided in (a) with the solvent system provided in (a) under conditions allowing for colorant removal but not allowing for dissolution of the polyalkylene terephthalate based polymer, thereby obtaining a solvent system, which is enriched in colorant compared to the solvent system provided, and a polymeric material, which is depleted in colorant compared to the colored polymeric material provided in (a);
   (c) depolymerisation or transesterification of the polymeric material, which is depleted in colorant obtained in (b); thereby obtaining monomers and/or oligomers or transesterification products.
2. The process of embodiment 1, wherein (b) comprises
   (b.1) providing the colored polymeric material in a first vessel and providing the solvent system in a second vessel;
      wherein first and second vessel are arranged spatially separated from each other but allowing a gaseous and fluid communication between each other;
   (b.2) forming a gaseous stream comprising at least parts of the solvent system, and removing the gaseous stream r from the second vessel;
   (b.3) allowing the solvent system to separate in liquid form from the gaseous stream at a position outside the second vessel (also outside the first vessel), obtaining a solvent system in liquid form;
   (b.4) contacting the colored polymeric material in the first vessel with the solvent system in liquid form, which had been separated from the gaseous stream in (b.3), thereby obtaining a solvent system, which is enriched in colorant and optionally in third polymer (iv) compared to the solvent system provided in (a), and a polymeric material, which is depleted in colorant and optionally in third polymer (iv) compared to the colored polymeric material provided in (a) and which comprises the polyalkylene terephthalate based polymer (i) and optionally the second polymer (iii).
3. The process according to embodiment 2, wherein (b.2) comprises
   (b.2.1) heating the solvent system to a temperature at least equal to or above the boiling temperature of the solvent system;
   (b.2.2) obtaining a gaseous stream comprising at least parts of the solvent system.
4. The process according to embodiment 2 or 3, wherein additionally an inert gas is applied, which forms parts of the gaseous stream comprising at least parts of the solvent system.
5. The process according to any one of embodiments 2 to 4, wherein (b.2) comprises
   (b.2.1′) heating the solvent system to a temperature T of at most 3K below the boiling temperature of the solvent system; so that a solvent system having elevated temperature is obtained;
   (b.2.2′) contacting the solvent system having elevated temperature with an inert gas, preferably passing inert gas through the solvent system having elevated temperature, so that a gaseous stream comprising at least parts of the solvent system is formed.
6. The process according to any one of embodiments 2 to 5, wherein (b.3) comprises
   (b.3.1) transferring the gaseous stream from the second vessel to a position outside the second vessel (also outside the first vessel);
   (b.3.2) allowing the parts of the solvent system comprised in the gaseous stream to separate in liquid form from the gaseous stream at the position outside the second vessel (also outside the first vessel), obtaining a solvent system in liquid form.
7. The process according to any one of embodiments 2 to 6, wherein (b.4) comprises
   (b.4.1) transferring the solvent system in liquid form obtained in (b.3.2) to the colored polymeric material in the first vessel;
   (b.4.2) contacting the colored polymeric material in the first vessel with the solvent system in liquid form,
      thereby obtaining a solvent system, which is enriched in colorant and optionally third polymer compared to the solvent system provided in (a), and a polymeric material, which is depleted in colorant and optionally in third polymer compared to the colored polymeric material provided in (a) and which comprises the polyalkylene terephthalate based polymer and optionally the second polymer.
8. The process according to any one of embodiments 2 to 7, wherein first vessel containing the polymeric material and second vessel containing the solvent system are arranged spatially separated from each other in that the first vessel containing the polymeric material is arranged above the second vessel containing the solvent system.
9. The process according to embodiment 7 or 8, wherein (b.4.2) comprises contacting the colored polymeric material with the solvent system in liquid form of (b.4.1) in that the solvent system in liquid form drops onto the colored polymeric material.
10. The process according to any one of embodiments 2 to 9, wherein the inert gas is selected from the group consisting of argon, helium, neon, nitrogen and mixtures of two or more of these inert gases, preferably comprises at least nitrogen, more preferably the inert gas is nitrogen.
11. The process according to any one of embodiments 2 to 10, wherein the inert gas is conducted through the solvent system and has a flow rate in the range of from 1 to 150 liter/h or is passed along the surface of the solvent system and has a flow rate of in the range of from 1 to 150 liter/h.
12. The process according to any one of embodiments 2 to 11, further comprising
   (b.5) removal of the solvent system obtained in (b.4) or (b.4.2), which is enriched in colorant and optionally in third polymer from the polymeric material (i.e. also out of the first vessel) and recirculation into the second vessel comprising the solvent system, preferably by dropping it into the second vessel comprising the solvent system (allowing the solvent system obtained in (b.4) or (b.4.2) or to drop into the second vessel comprising the solvent system).
13. The process according to any one of embodiments 2 to 12, wherein (b.2), (b.3) and (b.4), preferably (b.2), (b.3), (b.4) and (b.5), are carried out in continuous mode.
14. The process according to any one of embodiments 2 to 13, further comprising
   (b.6) at least partial removal of polymeric material obtained in (b.4) or (b.4.2), thereby obtaining a polymeric material which is depleted in colorant and optionally in third polymer (iv) compared to the colored polymeric material provided in (a) and which comprises the polyalkylene terephthalate based polymer and optionally the second polymer.
15. The process according to embodiment 14, further comprising
   (b.7) at least partial replacement of the polymeric material removed in (b.6) by fresh colored polymeric material without replacement of the solvent system provided in (a), wherein the fresh colored polymeric material comprises polyalkylene terephthalate based polymer, colorant, optionally second polymer and optionally third polymer, and repetition of step(s) (b.2), (b.3) and (b.4), optionally (b.5);
   wherein
   steps (b.6) and (b.7) are preferably repeated at least one further time, preferably in the range of one to at least 10 further time(s) (batch mode); or
   wherein steps (b.6) and (b.7) are carried out continuously (purge mode).
16. The process according to any one of embodiments 2 to 15, wherein in the range of from 1 to 50 weight-% of additional fresh solvent system, based on the total weight of the solvent system provided in (a) being 100 weight-% are added, wherein the addition is carried out discontinuously (batch mode) or continuously (purge mode).
17. The process according to embodiment 1, wherein (b) comprises
   (b.1′) contacting the colored polymeric material of (a) with a solvent system at a temperature T, which is <170° C., thereby obtaining a solvent system, which is enriched in colorant and optionally in third polymer (iv) compared to the solvent system provided in (a), and a polymeric material, which is depleted in colorant and optionally in third polymer (iv) compared to the colored polymeric material provided in (a);
   (b.2′) optionally separating the polymeric material, which is depleted in colorant and optionally in third polymer (iv), and which comprises the polyalkylene terephthalate based polymer, from the solvent system.
18. The process of embodiment 17, wherein contacting in (b.1′) is done at a temperature T, which is in the range of from 10 to <170° C., preferably in the range of from 110 to 165° C., more preferably in the range of from 120 to 150° C.
19. The process of embodiment 17 or 18, wherein (b.1′) is done at a pressure in the range of from 800 to 200,000 hPa.
20. The process according to any one of embodiments 1 to 19, wherein the solvent system comprises one or more solvent(s), wherein the solvent system
   (s.1) has Hansen solubility parameters with respect to
      energy from dispersion forces between molecules (δDₛₛ),
      energy from dipolar intermolecular force between molecules (δPₛₛ) and
      energy from hydrogen bonds between molecules (δHₛₛ),
      which fulfill equitation 1

(11)²≥4(δDₛₛ−17.5)²+(δPₛₛ−7.5)²+(δHₛₛ−7.5)² [equitation 1].
21. The process according to any one of embodiment 1 to 20, wherein the solvent system comprises one or more solvent(s), wherein the solvent system
   (s.1a) has Hansen solubility parameters with respect to
      energy from dispersion forces between molecules (δDₛₛ),
      energy from dipolar intermolecular force between molecules (δPₛₛ) and
      energy from hydrogen bonds between molecules (δHₛₛ), which fulfill equitation 2

(8.8)²≥4(δDₛₛ−20)²+(δPₛₛ−11.8)²+(δHₛₛ−4.5)² [equitation 2].
22. The process according to any one of embodiment 1 to 21, wherein
   (s.2) each solvent of the solvent system has a boiling point at 1013 hPa of at least 150° C., preferably of at least 160° C.
23. The process according to any one of embodiments 1 to 22, wherein
   (s.3) solvents having a functional group selected from the group consisting of hydroxyl (OH), amino (NH₂), carboxyl (COOH), and thiol (SH) are excluded.
24. The process according to any one of embodiments 1 to 23, wherein at least 90 weight-%, preferably at least 95 weight-%, more preferably at least 98 weight-%, more preferably in the range of from 99 to 100 weight-%, of the solvent system consist of two or more solvent(s), based on the total weight of the solvent system being 100 weight-%.
25. The process according to any one of embodiments 1 to 24, wherein at least 90 weight-%, preferably at least 95 weight-%, more preferably at least 98 weight-%, more preferably in the range of from 99 to 100 weight-%, of the solvent system consist of one solvent, which fulfills equitation 1 and/or 2, based on the total weight of the solvent system being 100 weight-%.
26. The process according to any one of embodiments 1 to 25, wherein the one or more solvent(s) of the solvent system is/are selected from the group consisting of N,N-dimethylbenzamide, N,N-dimethylphenylacetamide, 1,4-benzoquinone, acetophenone, dimethyl terephthalate, 1,3,5-trimethoxybenzene, 2-phenylacetophenone, N-methylcaprolactam, methylbenzoate, methyl-4-methoxybenzoate, butylene carbonate, propylene-glycol-dibenzoate, N-ethylpyrrolidone, benzophenone, di-benzyl malonate, N-ethyl-caprolactam, methyl 2-(5-oxotetrahydrofuran-3-yl)acetate (FAME), propiophenone, N-methoxypropyl-pyrrolidone, 1,4-cyclohexanedione, cyclohexane-carbonate, N-methoxyethyl-pyrrolidone, N,N-diethylphenylacetamide, phenyl acetate, 1-(2-hydroxyethyl) pyrrolidin-2-one acetate (HEPAc), N,N-diethylbenzamide, isopropyl-benzoate, cyclohexyl phenyl ketone, phenylacetic acid ethylester, phenylacetat, N-methyl-morpholine, benzyl-propionate, benzylacetate, Neopentyl-glycol-dibenzoate, tetrahydrofurfuryl acetate, N-methylimidazole, benzyl butyrate, 2-pyrrolidone, 2-phenoxyethanol propionate, 2-phenoxyethyl isobutyrate, N, N-dipropylbenzamide, N, N-dimethylacetamide, N, N-diethylacetamide, dihydrolevoglucosenon (Cyrene), propylenecarbonate, caprolactone, dimethylisosorbide, Nbutylpyrrolidone, t-butylpyrrolidone, methyl-1-methyl-5-oxopyrrolidine-3-carboxylate (MMOC), gamma-valerolactone (GVL), delta-valerolactone, gamma butyrolactone, methyl 5-(dimethylamino)-2-methyl-5-oxopentanoate (Rhodiasolv Polarclean), caprolactam, phen ethyl acetate, methyl phenylacetate, benzyl benzoate, N,N-dimethyllactamide (Agnique AMD 3L), and dimethyl sulfoxide (DMSO).
27. The process according to any one of embodiments 1 to 26, wherein the one or more solvent(s) of the solvent system is/are selected from the group consisting of dihydrolevoglucosenon (Cyrene), propylenecarbonate, caprolactone, dimethylisosorbide, N-butylpyrrolidone, t-butylpyrrolidone, methyl-1-methyl-5-oxopyrrolidine-3-carboxylate (MMOC), gamma-valerolactone (GVL), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, propyl benzoate, and dimethyl sulfoxide (DMSO).
28. The process according to any one of embodiments 1 to 27, wherein the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, propyl benzoate and GVL.
29. The process according to any one of embodiments 1 to 28, wherein the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), delta-valerolactone, gamma butyrolactone, methyl 5-(dimethylamino)-2-methyl-5-oxopentanoate (Rhodiasolv®Polarclean), caprolactam, phenethyl acetate, methyl phenylacetate, benzyl benzoate, phenyl benzoate, methyl benzoate, and propyl benzoate.
30. The process according to any one of embodiments 1 to 29, wherein the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), methyl 5-(dimethylamino)-2-methyl-5-oxopentanoate (Rhodiasolv®Polarclean), phenethyl acetate, and GVL.
31. The process according to any one of embodiments 1 to 30, wherein the one or more solvent(s) of the solvent system is/are selected from the group consisting of propylenecarbonate, N-butylpyrrolidone, t-butylpyrrolidone, methyl-1-methyl-5-oxopyrrolidine-3-carboxylate (MMOC), methyl 5-(dimethylamino)-2-methyl-5-oxopentanoate (Rhodiasolv®Polarclean), and phenethyl acetate.
32. The process of any one of embodiments 1 to 31, wherein the one or more solvent(s) is/are selected from the group consisting of gamma valerolactone (GVL), N-butylpyrrolidone (NBP), propylenecarbonate, acetophenone dimethyl sulfoxide (DMSO), dihydrolevoglucosenon (Cyrene), cyclohexanone and mixtures of two or more thereof, or from the group consisting of N-butylpyrrolidone (NBP), propylenecarbonate, acetophenone dimethyl sulfoxide (DMSO), dihydrolevoglucosenon (Cyrene), cyclohexanone and mixtures of two or more thereof.
33. The process of any one of embodiments 1 to 32, wherein the one or more solvent(s) is/are selected from the group consisting of GVL, NBP, propylenecarbonate, acetophenone, DMSO and mixtures of two or more thereof, or from the group consisting of NBP, propylenecarbonate, acetophenone, DMSO and mixtures of two or more thereof.
34. The process of any one of embodiments 1 to 33, wherein the one or more solvent(s) is/are selected from the group consisting of GVL, NBP, propylenecarbonate, acetophenone and mixtures of two or more thereof, or from the group consisting of NBP, propylenecarbonate, acetophenone and mixtures of two or three thereof.
35. The process of any one of embodiments 1 to 34, wherein the one or more solvent(s) comprise at least N-butylpyrrolidone (NBP), preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are NBP, based on a total weight of the one or more solvent(s) being 100 weight-%, more preferably the one solvent is NBP; or
   wherein the one or more solvent(s) comprise at least propylenecarbonate, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are propylenecarbonate, based on a total weight of the one or more solvent(s) being 100 weight-%, more preferably the one solvent is propylenecarbonate; or
   wherein the one or more solvent(s) comprise at least acetophenone, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are acetophenone, based on a total weight of the one or more solvent(s) being 100 weight-%, more preferably the one solvent is acetophenone; or
   wherein the one or more solvent(s) comprise at least dimethyl suolfoxide (DMSO), preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are DMSO, based on a total weight of the one or more solvent(s) being 100 weight-%, more preferably the one solvent is DMSO; or
   wherein the one or more solvent(s) comprise at least dihydrolevoglucosenon (Cyrene), preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are Cyrene, based on a total weight of the one or more solvent(s) being 100 weight-%, more preferably the one solvent is Cyrene; or
   wherein the one or more solvent(s) comprise at least cyclohexanone, preferably at least 90 weight-% of the one or more solvent(s), more preferably at least 95 weight-%, more preferably at least 98 weight-%, more preferably at least 99 weight-%, more preferably at least 99.5 weight-%, more preferably at least 99.9 weight-%, of the one or more solvent(s) are cyclohexanone, based on a total weight of the one or more solvent(s) being 100 weight-%, more preferably the one solvent is cyclohexanone.
36. The process of any one of embodiments 1 to 35, wherein ethyl benzoate and butyl benzoate are excluded as solvent(s).
37. The process according to any one of embodiments 1 to 36, wherein the solvent system comprises GVL, wherein preferably at least 90 weight-%, more preferably at least 95 weight-%, more preferably at least 98 weight-%, more preferably in the range of from 99 to 100 weight-%, based on the total weight of the solvent system being 100 weight-%, of the solvent system of the solvent system consist of GVL.
38. The process of any one of embodiments 1 to 37, wherein the depolymerisation of (c) is done by a method selected from the group consisting of alcoholysis, glycolysis, hydrolysis, aminolysis, ammonolysis, and mixtures of two or more of these methods.
39. The process of embodiment 38, wherein the depolymerisation of (c) is done by alcoholysis, wherein (c) comprises contacting the polymeric material depleted in colorant obtained in (b) with a C₁ to C₄ alkyl mono alcohol, thereby obtaining a di (C₁ to C₄ alkyl) terephthalate and/or a di (C₁ to C₄ alkyl) isophthalate and ethylene glycol and/or cyclohexanedimethanol.
40. The process of embodiment 39, wherein the C₁ to C₄ alkyl mono alcohol is methanol, wherein di methyl terephthalate and/or di methyl isophthalate and ethylene glycol and/or cyclohexanedimethanol are obtained.
41. The process of embodiment 37, wherein the depolymerisation of (c) is done by glycolysis, wherein (c) comprises contacting the polymeric material depleted in colorant obtained in (b) with a C₁ to C₆ alkyl diol, thereby obtaining a bis(hydroxy C₁ to C₆ alkyl) terephthalate and/or bis(hydroxy C₁ to C₆ alkyl) isophthalate and ethylene glycol and/or cyclohexanedimethanol.
42. The process of embodiment 41, wherein the C₁ to C₆ alkyl diol is ethylene glycol, wherein bis(hydroxy ethylene) terephthalate and/or bis(hydroxy ethylene) isophthalate and and/or bis(hydroxy C₁ to C₆ alkyl) isophthalate and ethylene glycol and/or cyclohexanedimethanol are obtained.
43. The process of embodiment 37, wherein the depolymerisation of (c) is done by hydrolysis, wherein (c) comprises contacting the polymeric material depleted in colorant obtained in (b) with water, thereby obtaining terephthalic acid (TPA) and/or isophthalic acid and ethylene glycol and/or cyclohexanedimethanol.
44. The process of embodiment 43, wherein the depolymerisation of (c) is done by alkaline hydrolysis.
45. The process of embodiment 43, wherein the hydrolysis is done by enzymatically catalyzed hydrolysis, wherein (c) comprises contacting the polymeric material depleted in colorant obtained in (b) with water in the presence of an enzyme, thereby obtaining terephthalic acid (TPA) and/or isophthalic acid and ethylene glycol and/or cyclohexanedimethanol.
46. The process of any one of embodiments 43 to 45, wherein the depolymerisation of (c) is done by hydrolysis at a pressure in the range of from 40 to 120 hPa.
47. The process of embodiment 37, wherein the depolymerisation of (c) is done by hydrolysis, wherein (c) comprises
   (c.1) preparing a melt of the polymeric material, which is depleted in colorant, thereby obtaining a melt of the polymeric material, which is depleted in colorant;
   (c.2) contacting the melt of the polymeric material obtained in (c.1) with a base, preferably an inorganic alkali metal based base, more preferably sodium hydroxide, potassium hydroxide or a mixture of sodium hydroxide and potassium hydroxide, thereby obtaining an alkali containing melt;
   (c.3) contacting the alkali containing melt obtained in (c.2) with water, thereby obtaining terephthalate (with a corresponding alkali metal cation) and/or isophthalate (with a corresponding alkali metal cation) and ethylene glycol and/or cyclohexanedimethanol.
48. The process of embodiment 37, wherein the depolymerisation of (c) is done by aminolysis, wherein (c) comprises contacting the polymeric material depleted in colorant obtained in (b) with a mono alkyl amine R¹R²NH₂, wherein R¹ is C₁ to C₄ alkyl and R² is selected from hydrogen atom and C₁ to C₄ alkyl; thereby obtaining R¹R²NC(═O)-phenyl-C(═O)NR²R¹, wherein the substituents at the phenyl ring are in para or meta position, and ethylene glycol and/or cyclohexanedimethanol.
49. The process of embodiment 37, wherein the depolymerisation of (c) is done by ammonolysis, wherein (c) comprises contacting the polymeric material depleted in colorant obtained in (b) with ammonia (NH₃), thereby obtaining H₂NC(═O)-phenyl-C(═O)NH₂, wherein the substituents at the phenyl ring are in para or meta position, and ethylene glycol and/or cyclohexanedimethanol; wherein the ammonia is provided solvent-free or dissolved in a solvent, wherein the solvent is preferably one or more of glycerin, glycol (preferably C₁ to C₆ alkyl diol), alcohol (preferably C₁ to C₄ alkyl mono alcohol).
50. The process of embodiment 1, wherein the transesterification of (c) is done by acid or base catalyzed reaction with an alcohol.
51. The process of embodiment 50, wherein one or more catalysts containing metal cations are used, wherein the metal is preferably titanium or tin.
52. The process of embodiment 50 or 51, wherein the alcohol is R³—OH, wherein R³ is a linear or branched C₂ to C₁₂ alkyl residue.
53. The process of embodiment 52, wherein the alcohol is selected from the group consisting of 2-ethylhexanol, iso-propanol, isononanol, pentanol, butanol, isomers of these alcohols, and mixtures of two or more thereof.
54. The process of any one of embodiments 1 to 53, wherein the polyalkylene terephthalate based polymer consists of either oxyethylen units or oxybutylen units and oxyterephthaloyl units, wherein in case of oxyethylen units, in the range of from 0 to 5 mol-% of the oxyterephthaloyl units are replaced by oxyisophthaloyl units and/or in the range of from 0 to 49 mol-% of the oxyethylen units are replaced by oxymethylene cyclohexylene methylene units; wherein more preferably, the polyalkylene terephthalate based polymer is selected from the group consisting of PET (polyethylene terephthalate), PETI (poly(ethylene terephthalate-co-isophthalate)), PBT (polybutylene terephthalate), and mixtures of two or three of these polymers; wherein more preferably the polyalkylene terephthalate based polymer comprises or is PET.
55. The process of any one of embodiment 1 to 54, wherein the third polymer (iv) is an elastic fiber, wherein the elastic fiber comprises, preferably consists of one or more polyurethane based elastic fiber(s) and/or one or more polyester based elastic fiber(s), more preferably the elastic fiber comprises, preferably consists of one or more polyurethane based elastic fiber(s), wherein more preferably at least 40 weight-%, more preferably at least 45 weight-%, more preferably at least 50 weight-%, more preferably at least 55 weight-%, more preferably at least 60 weight-%, more preferably at least 65 weight-%, more preferably at least 70 weight-%, more preferably at least 75 weight-%, more preferably at least 80 weight-%, more preferably at least 85 weight-%, more preferably at least 90 weight-%, more preferably at least 95 weight-%, more preferably at least 99.9 weight-%, of the elastic fiber are polyurethane based elastic fiber(s), each based on the total weight of the elastic fiber being 100 weight-%.
56. The process according to any one of embodiments 1 to 55, wherein the colorant is selected from the group consisting of dye and optical brightener and mixtures of dye and optical brightener.
57. The process according to any one of embodiments 1 to 56, wherein depleted in colorant regarding the polymeric material obtained means that the L*a*b* values of the polymeric material, which is depleted in colorant compared to the polymeric material provided in (a), change in that:
   the absolute value of a* decreases, preferably by at least 0.2; and/or, preferably and,
   the absolute value of b* changes, preferably by at least 0.2; and/or, preferably and,
   the L* value increases, preferably by at least 4,
   each compared to the L*a*b* values of the colored polymeric material provided in (a), wherein L*a*b* values are determined according to DIN 5033 and DIN EN ISO 11664-1.6.
58. The process according to any one of embodiments 1 to 57, further comprising, if second polymer (iii) was present in the polymeric material provided in (a):
   (d) separating the second polymer (iii) from the monomers and/or oligomers or transesterification products obtained in (c), thereby obtaining separate monomers and/or oligomers or transesterification products and separate second polymer (iii).
59. The process according to any one of embodiments 17 to 58, comprising
   (b.x) optionally washing the polymeric material removed in (b.6) or separated in (b.2′) with a washing solvent, thereby obtaining a washed polymeric material, which is depleted in colorant compared to the polymeric material provided in (a).
60. The process according to any one of embodiments 14 or 17 to 59, comprising
   (b.y) drying the polymeric material removed in (b.6) or separated in (b.2′) and/or the washed polymeric material obtained in (b.x).
61. The process according to any one of embodiments 1 to 60, further comprising, if third polymer (iv) was present in the polymeric material provided in (a):
   (b.z) separating the third polymer (iv) from the solvent system, which is enriched in colorant obtained in (b), (b.4), (b.4.2.), (b.5) and/or (b.2′), thereby obtaining separate third polymer (iv).
62. Monomer, oligomer or transesterification product of a polyalkylene terephthalate based polymer obtained or obtainable by the process of any one of embodiments 1 to 61, preferably of step (d)
63. Process, preferably according to any one of embodiments 1 to 61, comprising the further step:
   converting, preferably polymerizing, the monomers, oligomers or transesterification products of a polyalkylene terephthalate based polymer obtained or obtainable according to any one of embodiments 1 to 61 to obtain one or more polymer or polymer product.
64. Process according to embodiment 63, wherein the one or more polymer is/are polyalkylene terephthalate based polymer, which consists of either oxyethylen units or oxybutylen units and oxyterephthaloyl units, wherein in case of oxyethylen units, in the range of from 0 to 5 mol-% of the oxyterephthaloyl units are replaced by oxyisophthaloyl units and/or in the range of from 0 to 49 mol-% of the oxyethylen units are replaced by oxymethylene cyclohexylene methylene units; wherein more preferably, the polyalkylene terephthalate based polymer is selected from the group consisting of PET (polyethylene terephthalate), PETI (poly(ethylene terephthalate-co-isophthalate)), PBT (polybutylene terephthalate), and mixtures of two or three of these polymers; wherein more preferably the polyalkylene terephthalate based polymer comprises or is PET.
65. Process according to embodiment 63 or 64, wherein the polymer product is a textile, a fiber, a packaging, a plastic, preferably:
   a part of a car; preferably cylinder head cover, engine cover, housing for charge air cooler, charge air cooler flap, intake pipe, intake manifold, connector, gear wheel, fan wheel, cooling water box, housing, housing part for heat exchanger, coolant cooler, charge air cooler, thermostat, water pump, radiator, fastening part, part of battery system for electromobility, dashboard, steering column switch, seat, headrest, center console, transmission component, door module, A, B, C or D pillar cover, spoiler, door handle, exterior mirror, windscreen wiper, windscreen wiper protection housing, decorative grill, cover strip, roof rail, window frame, sunroof frame, antenna panel, headlight and taillight, engine cover, cylinder head cover, intake manifold, airbag, cushion, or coating;
   a cloth; preferably shirt, trousers, pullover, boot, shoe, shoe sole, tight or jacket;
   an electrical part; preferably electrical or electronic passive or active component, circuit board, printed circuit board, housing component, foil, line, switch, plug, socket, distributor, relay, resistor, capacitor, inductor, bobbin, lamp, diode, LED, transistor, connector, regulator, integrated circuit (IC), processor, controller, memory, sensor, microswitch, microbutton, semiconductor, reflector housing for light-emitting diodes (LED), fastener for electrical or electronic component, spacer, bolt, strip, slide-in guide, screw, nut, film hinge, snap hook (snap-in), or spring tongue;
   a consumer, agricultural product or pharmaceutical product; preferably tennis string, climbing rope, bristle, brush, artificial grass, 3D printing filament, grass trimmer, zipper, hook and loop fastener, paper machine clothing, extrusion coating, fishing line, fishing net, offshore line and rope, vial, syringe, ampoule, bottle, sliding element, spindle nut, chain conveyor, plain bearing, roller, wheel, gear, roller, ring gear, screw and spring dampers, hose, pipeline, cable sheathing, socket, switch, cable tie, fan wheel, carpet, box or bottle for cosmetics, mattress, cushion, insulation, detergent, dishwasher tabs or powder, shampoo, body wash, shower gel, soap, fertilizer, fungicide, or pesticide;
   a packaging for the food industry; preferably mono- or multi-layer blown film, cast film (mono- or multi-layer), biaxially stretched film, or laminating film; or
   a part of a construction; preferably a rotor blade, insulating material, frame, housing, wall, coating, or separating wall.
66. Process, preferably according to any one of embodiments 1 to 61, comprising the further step:
   converting the separated second polymer (iii) and/or the separated third polymer (iv) obtainable by or obtained by the process of any one of embodiments 1 to 64, preferably the separated second polymer (iii) obtained or obtainable from step (d) and/or the separated third polymer (iv) obtained or obtainable from any one of steps ((b), (b.4), (b.4.2.), (b.5) and (b.2′), to obtain one or more monomer, polymer or polymer product.
67. Process according to embodiment 66,
   wherein the polymer is and/or the polymer product comprises polyamide (PA); preferably PA 6 or PA 66; polyisocyanate polyaddition product; preferably polyurethane (PU), thermoplastic polyurethane (TPU), polyurea or polyisocyanurate (PIR); low-density polyethylene (LDPE), high-density polyethylene (HDPE), polyethylene (PE), polypropylene (PP), polyvinyl chloride (PVC), polyvinyl acetate (PVA), polystyrene (PS), poly acrylonitrile butadiene styrene (ABS), poly styrene acrylonitrile (SAN), poly acrylate styrene acrylonitrile (ASA), polytetrafluoroethylene (PTFE), poly(methyl acrylate) (PMA), poly(methyl methacrylate) (PMMA), polybutadiene (BR, PBD), poly(cis-1,4-isoprene), poly(trans-1,4-isoprene), polyoxymethylene (POM), polyethylene terephthalate (PET), polybutylene terephthalate (PBT), polybutylene adipate coterephthalate (PBAT), polyester (PES), polyether sulfone (PESU), polyhydroxyalkanoate (PHA), poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), polylactic acid (PLA), polysulfone (PSU), polyphenylene sulfone (PPSU), polycarbonate (PC), polyether ether ketone (PEEK), poly(p-phenylene oxide) (PPO), poly(p-phenylene ether) (PPE); or copolymer or mixture thereof.
68. Process according to embodiment 66 or 67,
   wherein the polymer and/or the polymer product is/are or is/are a part of:
      a part of a car; preferably cylinder head cover, engine cover, housing for charge air cooler, charge air cooler flap, intake pipe, intake manifold, connector, gear wheel, fan wheel, cooling water box, housing, housing part for heat exchanger, coolant cooler, charge air cooler, thermostat, water pump, radiator, fastening part, part of battery system for electromobility, dashboard, steering column switch, seat, headrest, center console, transmission component, door module, A, B, C or D pillar cover, spoiler, door handle, exterior mirror, windscreen wiper, windscreen wiper protection housing, decorative grill, cover strip, roof rail, window frame, sunroof frame, antenna panel, headlight and taillight, engine cover, cylinder head cover, intake manifold, airbag, cushion, or coating;
      a cloth; preferably shirt, trousers, pullover, boot, shoe, shoe sole, tight or jacket;
      an electrical part; preferably electrical or electronic passive or active component, circuit board, printed circuit board, housing component, foil, line, switch, plug, socket, distributor, relay, resistor, capacitor, inductor, bobbin, lamp, diode, LED, transistor, connector, regulator, integrated circuit (IC), processor, controller, memory, sensor, microswitch, microbutton, semiconductor, reflector housing for light-emitting diodes (LED), fastener for electrical or electronic component, spacer, bolt, strip, slide-in guide, screw, nut, film hinge, snap hook (snap-in), or spring tongue;
      a consumer, agricultural product or pharmaceutical product; preferably tennis string, climbing rope, bristle, brush, artificial grass, 3D printing filament, grass trimmer, zipper, hook and loop fastener, paper machine clothing, extrusion coating, fishing line, fishing net, offshore line and rope, vial, syringe, ampoule, bottle, sliding element, spindle nut, chain conveyor, plain bearing, roller, wheel, gear, roller, ring gear, screw and spring dampers, hose, pipeline, cable sheathing, socket, switch, cable tie, fan wheel, carpet, box or bottle for cosmetics, mattress, cushion, insulation, detergent, dishwasher tabs or powder, shampoo, body wash, shower gel, soap, fertilizer, fungicide, or pesticide;
      a packaging for the food industry; preferably mono- or multi-layer blown film, cast film (mono- or multi-layer), biaxially stretched film, or laminating film; or
      a part of a construction; preferably a rotor blade, insulating material, frame, housing, wall, coating, or separating wall.
69. Process according to embodiment 66 or 67,
   wherein the content of the separated second polymer (iii) and/or the separated third polymer (iv) in the polymer and/or polymer product is 1 weight-% or more, preferably 2 weight-% or more, more preferably 5 weight-% or more, more preferably 15 weight-% or more, more preferably 30 weight-% or more, more preferably 40 weight-% or more, more preferably 60 weight-% or more, more preferably 80 weight-% or more, more preferably 90 weight-% or more, more preferably 95 weight-% or more; and/or
   wherein the content of the separated second polymer in the polymer and/or polymer product is 100 weight-% or less, preferably 95 weight-% or less, more preferably 90 weight-% or less, more preferably 50 weight-% or less, more preferably 25 weight-% or less, more preferably 10 weight-% or less; and
   preferably wherein the content is determined based on identity preservation and/or segregation and/or mass balance and/or book and claim chain of custody models, preferably based on mass balance, preferably the International Sustainability and Carbon Certification (ISCC) standard.

The present invention is further illustrated by the following reference examples, comparative examples, and examples.

### EXAMPLES

### Methods for Analytics:

### GPC (Gel-Permeation Chromatography):

### Sample Preparation:

7.5 mg sample was dissolved in 5 ml eluent (hexafluorisopropanol+0.05 weight-% trifluoro potassium acetate) over night. All sample solutions were filtered by a Millipore Millex FG (0.2 μm) filtered prior to injection. Sealed sample vials were placed into the auto sampler.

### Experimental Conditions:

An Agilent 1100 HPLC system, consisting of an isocratic pump, vacuum degasser, auto sampler and a column oven (40° C.) was used. Furthermore, the Agilent system contained as detectors a Differential Refractive Index (DRI) and a variable Ultra Violet (UVW) Detector. Data acquisition and data processing of conventionally SEC data were done by WinGPC Unichrom, of PSS (Polymer Standard Services). A combination of a PL-HFIP guard (7.5×50 mm) column and 2 PLHFIP Gel columns (7.5×300 mm, 9u) of Agilent were put in series. As an eluent, hexafluorisopropanol+0.05 weight-% trifluoro potassium acetate was used for the initially provided material, whereas+0.1 weight-% trifluoro potassium acetate was used for the samples after treatment, for each with a flow rate of 1 ml/min. Of each sample solution 50 μl was injected. The calibration was obtained by narrow molar mass distributed PMMA standards (Polymer Standard Services) having a molar mass range of M=800 till M=2.200.000 g/mol for the initially provided material and M=596 till M=2.050.00 g/mol for the samples after treatment respectively. Molar masses outside this range were extrapolated.

### CIE-LAB:

L*a*b* values were determined in that the samples were measured using an integrating sphere and UV/VIS-remission spectra (with a wavelength area of 400-700 nm) were obtained. The data of these spectra were analyzed by the software OptLab-SPX using 2° standard observer and the standard light type C. The OptLab-SPX software calculates the L*a*b*-values based on DIN 5033 and DIN EN ISO 11664-1.6 from the years 2007-2014.

### GC Area %:

The sample was analyzed by gas chromatography (GC), wherein the method detected individual components from a sample dependent on their individual retention times. The concentration of the individual component in the sample were given in its percental peak area as GC-area %.

### Chemicals

| | |
|---|---|
| (Chemical) name | Abbreviation |

Polymeric materials

| | |
|---|---|
| polyethylene terephthalate | PET |
| block copolymer comprising polyurethane and polyethylene | Elastane (Spandex) |
| glycol or pTHF (≥85 weight-% polyurethane) | |

solvents

| | |
|---|---|
| 2-ethylhexanol | — |
| ethylene glycol | — |
| water | — |
| acetone | — |
| gamma-valerolactone | GVL |
| sodium hydrogen carbonate | NaHCO₃ |

### Reference Example 1: General Procedure for Single Use of Solvent System/Colorant Removal Via Gaseous Stream

10 g of colored and optionally elastic fiber containing polymeric material (in any processing form, e.g. shredded/cut textile, flakes etc.) was placed in a basket with holes which was placed on a reaction vessel, (e.g. flask), especially above an opening thereof, in which a solvent system was added (in mass-based ratio solvent:polymeric material 100:1 to 1:1, preferred 80:1 to 10:1), wherein the basket and especially the material contained therein did not come into direct contact with the solvent system but a gaseous and also liquid communication between basket and solvent system in reaction vessel was possible. The solvent system was heated in the reaction vessel by use of a suitable heating system (e.g. oil bath, heating blocks, mini-plant vessels) (i) to a temperature at least equal to or above the boiling temperature of the solvent system under inert gas atmosphere or (ii) to a temperature in the range of from 120° C. to at most 3K below the boiling temperature of the solvent and brought into contact with an inert gas, having a flow rate of, for example, 24 l/h, so that a gaseous phase comprising solvent system was formed and the gaseous phase was allowed to come into contact with the colored polymeric material in the basket, combined with and/or followed by a condensation (including also a separation of solvent contained in liquid form in the gaseous phase) of the solvent system at a cooler's surface, wherein the condensed solvent system, which comprises parts of the colorant, was allowed to flow back into the reaction vessel comprising the solvent system. Due a continuous inert gas flow (N₂) and the steam/condensate of solvent system being formed the colored optionally elastic fiber containing polymeric material in the basket above was discolored and optionally the elastic fiber was removed. After 0.1-8 h the system was cooled down and the discolored and optionally elastic fiber-depleted polymeric material could be collected, whereby solvent system enriched with colorant and optionally elastic fiber was obtained in a separate reaction vessel. For an easy removal of solvent system and a faster drying process of the discolored and optionally elastic fiber-depleted polymeric material pieces, small amounts of acetone could be used in an optional washing step. The thus obtained discolored and optionally elastic fiber-depleted polymeric material pieces were dried (for example in a vacuum compartment dryer).

The samples were analysed before the initial discoloration step (colored and optionally elastic fiber containing polymeric material), and after the final drying step (recycled and re-obtained colourless and optionally elastic fiber-depleted PET solid). In case of colour removal L*a*b* values were determined

### Examples 1 to 5: Discoloration of PET by Treatment with Gaseous and Re-Condensed Solvent System

Polymeric materials were treated according to the procedure of Reference Example 1, option (ii), the results are listed in Table 1 below:

TABLE 1
Overview material and results of Examples 1 to 5 according
to the procedure of Reference Example 1, option (ii).

| | | | | L*a*b* value | |
|---|---|---|---|---|---|
| | | Polymeric | | Before | After |
| Example No. | solvent system | material | Color | treatment | discoloration |
| 1 | GVL | PET textile | Yellow | L* = 78.2 | L* = 86 |
| | | | | a* = −10.8 | a* = −1 |
| | | | | b* = 42.3 | b* = 6 |
| 2 | GVL | PET textile | Green | L* = 54.8 | L* = 86 |
| | | | | a* = −32.3 | a* = −1 |
| | | | | b* = 21.2 | b* = 6 |
| 3 | GVL | PET textile | Blue | L* = 36.9 | L* = 85 |
| | | | | a* = 1.0 | a* = 0 |
| | | | | b* = −6.0 | b* = 5 |
| 4 | GVL | PET textile | Black | L* = 35.8 | L* = 81 |
| | | | | a* = 0.1 | a* = 1 |
| | | | | b* = −0.8 | b* = 7 |
| 5 | GVL | PET textile | Black | L* = 36.9 | L* = 88 |
| | | | (different | a* = −0.8 | a* = 0 |
| | | | source) | b* = −2.3 | b* = 2 |

For the green PET textile of Example 2 and the black PET textile of Example 5, molecular weights (Mn, Mw) and dispersity were determined based on GPC before and after the treatment. The results are shown below in Table 2.

TABLE 2
Molecular weights and dispersity of green and
black textile before and after treatment (GPC)

| | |
|---|---|
| Before treatment | After treatment |

Green textile of Example 2

| | | |
|---|---|---|
| number average molecular | 16900 | 25800 |
| weight Mn [g/mol] | | |
| Mass average molecular | 45100 | 49500 |
| weight Mw [g/mol] | | |
| Dispersity Mw/Mn | 2.7 | 1.9 |

Black textile of Example 5

| | | |
|---|---|---|
| number average molecular | 16800 | 29400 |
| weight Mn [g/mol] | | |
| Mass average molecular | 48500 | 55700 |
| weight Mw [g/mol] | | |
| Dispersity Mw/Mn | 2.9 | 1.9 |

It was found that the solvent system, here GVL used as solvent system, used with a gaseous stream combined with re-condensing and/or separation in liquid form, effectively removed the colorant from the polymeric material, here PET textiles of different colors, without harming at least the number average molecular weight Mn of the re-obtained PET—the Mn of the re-obtained PET was always greater or at least equal to the Mn of the PET comprised in the material initially provided. The same applies even for the mass average molecular weight Mw, which was also always greater or at least equal after treatment. The re-obtained PET had a dispersity Mw/Mn (Mass average molecular weight Mw divided by number average molecular weight Mn) in the range of from 60 to 95%, preferably in the range of from 75 to 90% of the dispersity Mw/Mn of the PET comprised in the material provided (100%).

### Example 6: Transesterifcation of PET to DOTP

A reactor equipped with a distillation column was filled with 2-ethylhexanol (8.0 equivalents, weight based), colour-depleted PET from Example 2 (1.0 equivalent, weight based) and Na—HCO₃ (1.25 weight-% compared to 100 weight-% of PET). The stirred mixture was heated to at most 220° C. while a mixture of 2-ethylhexanol and ethylene glycol was continuously distilled off. The equivalent amount of fresh 2-ethylhexanol compared to what was distilled off was added to the reaction mixture. The distillation was continued until the distillate did not contain any ethylene glycol anymore, determined by measurement of density. The excess of 2-ethylhexanol were distilled off and the raw product dioctyl terephthalate DOTP, bis(2-ethylhexyl) terephthalate) was separated by filtration (e.g. heated pressure filtration at 120° C.), optionally with filtration aids.

Purity of DOTP (GC area %): 99.85%

### Reference Example 2: Colorant Removal in Solution

PET textile (of any colour) was cut/shredded into pieces and placed in a reaction vessel (e.g. flask, tube, reaction vessel). The solvent system was added (in mass-based ratio solvent:polymeric material 100:1 to 1:1, preferred 10:1-1:1) and the mixture was heated by use of a suitable heating system (e.g. oil bath, heating blocks, mini-plant vessels) to a temperature in the range of from 60 to 160° C. After 0.5-8 h the mixture was filtered, whereby the solvent enriched in colorant and discolored polymeric material was obtained and the colour-depleted polymeric material was washed with a small amount of the respective solvent. For an easy removal of said solvent and a faster drying process of the colour-depleted polymeric material pieces, small amounts of acetone can be used in a second washing step. The thus obtained colour-depleted polymeric material were dried (for example in a vacuum compartment dryer).

### Example 7: Discoloration of PET by Treatment with Solvent System in Solution

10 g of a black PET textile was treated according to the procedure of Reference Example 2 with GVL as solvent at 150° C. for 4 hours. L*a*b* values before and after treatment were determined. The results are listed in Table 3 below:

TABLE 3
Overview material and results of Example 7 - L*a*b* values

| | | | | L*a*b* value | |
|---|---|---|---|---|---|
| Example | solvent | Polymeric | | | after treatment |
| No. | system | material | Color | Before treatment | (discoloration) |
| 7 | GVL | PET | Black | L* = 36.9 | L* = 88 |
| | | textile | | a* = −0.7 | a* = 0.9 |
| | | | | b* = −2.3 | b* = 2.2 |

### Example 8: Depolymerization

A mixture of 0.5 g previously discolored PET textile flakes from Example 7, 4.25 g ethanol, 0.75 g deionized water and 0.23 g sodium hydroxide were filled in a glass vessel with a magnetic stirrer and heated to 160° C. by microwave irradiation for 1 h. The white suspension was allowed to cool to ambient temperature, filtered and washed with ethanol (2×2 ml). The obtained white solid was redissolved in 10 g of hot water and filtered, yielding a colourless, clear solution. The filtrate was furthermore treated with 0.5 g of concentrated sulfuric acid, leading to a precipitation of a white, fine powder. The obtained solid was removed by filtration, dried in vacuo at 80° C. over night to yield terephthalic acid as a white solid (0.36 g, yield 96%). An 1H NMR spectrum of the product was measured and showed no impurities (FIG. 1).

### Comparative Example 1: Discoloration According to Prior Art

US 2016/0009893 A1 describes a color removal with ethylene glycol (EG). 10 g of a black PET textile was cut/shredded into pieces and was placed in a reaction vessel (e.g. flask, tube, reaction vessel). 100 mL of ethylene glycol were added, and the mixture was heated by use of a suitable heating system (e.g. oil bath, heating blocks, mini-plant vessels) to a temperature of 150° C. After 4 h the mixture was filtered, and the polymeric material was washed with a small amount of ethylene glycol. For an easy removal of ethylene glycol and a faster drying process of the polymeric material pieces, small amounts of acetone can be used in a second washing step. The thus obtained polymeric material was dried (for example in a vacuum compartment dryer). L*a*b* values before and after treatment were determined, as well as molecular weights and dispersity before and after treatment were determined by GPC. The results are listed in Tables 4 and 6 below:

TABLE 4
Overview material and results of Comparative Example 1 - L*a*b* values

| | | | | L*a*b* value | |
|---|---|---|---|---|---|
| Example | solvent | Polymeric | | | after treatment (no |
| No. | system | material | Color | Before treatment | discoloration) |
| 7 | GVL | PET | Black | L* = 36.9 | L* = 54 |
| | | textile | | a* = −0.7 | a* = 0 |
| | | | | b* = −2.3 | b* = 1 |

TABLE 5
Overview material and results of Comparative Example 1-GPC

| | | | | number | | Mass | | | |
|---|---|---|---|---|---|---|---|---|---|
| | | | | average | | average | | | |
| | | | | molecular | | molecular | | | |
| Example | solvent | Polymeric | | weight Mn | | weight Mw | | Dispersity | |
| No. | system | material | Color | [g/mol] | | [g/mol] | | Mw/Mn | |
| 7 | GVL | PET tex- | Black | Before | After | Before | after | Before | after |
| | | tile | | 17300 | 15700 | 48600 | 38200 | 2.8 | 2.4 |

It was found that ethylene glycol was not able to discolor PET as apparent from the L*a*b* values of Example 7 (Table 2) versus the L*a*b* values of Comparative Example 1 (Table 4). Furthermore, trying to use ethylene glycol as discoloration solvent resulted in a significant decrease in molecular mass average of PET.

### Examples 9 to 12: Discoloration of PET by Treatment with Gaseous and Re-Condensed Solvent System

Polymeric materials were treated according to the procedure of Reference Example 1, option (i), the results are listed in Table 6 below:

TABLE 6
Overview material and results of Examples 9, 10, 11 and 12 according
to general procedure of Reference Example 1, option (i).

| | | | | L*a*b* value | |
|---|---|---|---|---|---|
| | Polymeric | solvent | | Before | After |
| Example No. | material | system | Color | treatment | discoloration |
| 9 | PET textile | GVL | Black | L* = 36.9 | L* = 84 |
| | | | | a* = −0.8 | a* = 0 |
| | | | | b* = −2.3 | b* = 2 |
| 10 | PET textile | Cyrene | Black | L* = 36.9 | L* = 89 |
| | | | | a* = −0.8 | a* = −1 |
| | | | | b* = −2.3 | b* = 2 |
| 11 | PET textile | DMSO | Black | L* = 36.9 | L* = 87 |
| | | | | a* = −0.8 | a* = −1 |
| | | | | b* = −2.3 | b* = 1 |
| 12 | PET textile | Cyclohexanon | Black | L* = 36.9 | L* = 87 |
| | | | | a* = −0.8 | a* = 0 |
| | | | | b* = −2.3 | b* = 4 |

It was found that using the solvent system as gaseous stream combined with re-condensing and/or separation in liquid form, effectively removed the colorant from the polymeric material, here PET textiles of different colors effectively.

### SHORT DESCRIPTION OF THE FIGURES

FIG. 1 shows the 1H NMR spectrum of the terephthalic acid obtained from depolymerization of Example 8 in DMSO-de (*).

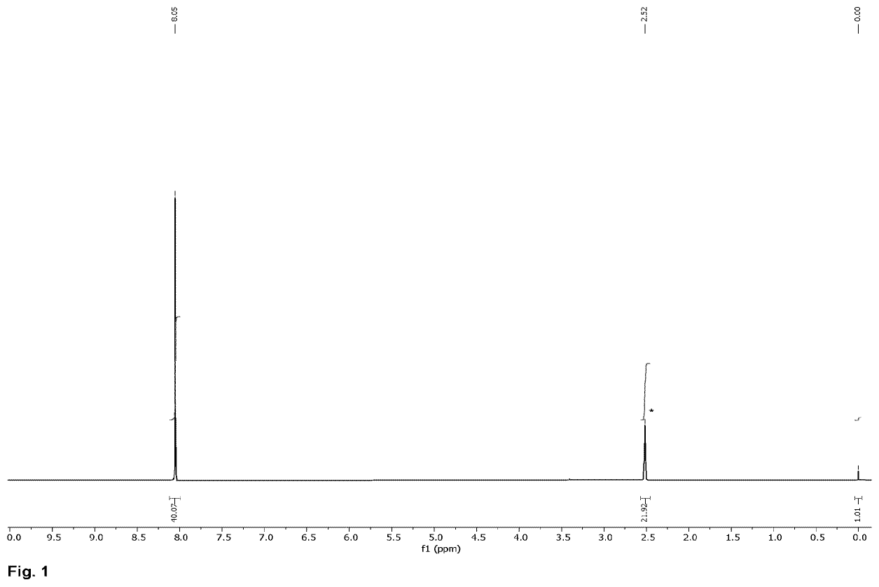

### CITED LITERATURE

Raheem et al., Journal of Cleaner Production 225 (2019) 1052e1064
Paszun et al., Ind. Eng. Chem. Res. 1997, 36, 1373
Barnard et al., Green Chem., 2021, 23, 3765-3789
EP 2 784 110 A1
U.S. Pat. No. 10,876,240 B2
DE 2223466 A1
“Dyes and Pigments” Metin Açikyildiz, Kübra Günes, Ahmet Gürses Springer, 2016 (ISBN: 10: 3319338900)
Industrial Organic Pigments-Klaus Hunger, Thomas Heber, Martin U. Schmidt, Friedrich Reisinger, Stefan Wanne Wiley-VCH, 4th edition, 2018 (ISBN: 978-3-527-32608-2)
Chemistry and Technology of Natural and Synthetic Dyes and Pigments-Ashis Kumar Samanta, Nasser Awwad, IntechOpen, 2020 (ISBN: 9781789859980, 9781789859973, 9781839687587)
Encyclopedia of Color, Dyes, Pigments—Volume 1, Gerhard Pfaff, de Gruyter, 2021 (ISBN: 311058588X)
Heinrich Zollinger: Color Chemistry: Syntheses, Properties, and Applications of Organic Dyes and Pigments. 3^{rd} edition. WILEY-VCH Verlag, Weinheim 2003 (ISBN: 3-906390-23-3)
Klaus Hunger (Ed.): Industrial Dyes: Chemistry, Properties, Applications. WILEY-VCH Verlag, Weinheim 2003 (ISBN: 3-662-01950-7)
Hermann Rath: Lehrbuch der Textilchemie. einschl. der textilchemischen Technologie. 2nd edition. Springer-Verlag, Berlin, Heidelberg 1963 (ISBN: 978-3-662-00065-6)
Wilfried Kratzert, Rasmus Peichert: Farbstoffe. Quelle & Meyer, Heidelberg 1981 (ISBN: 3-494-01021-8)
Ullmann's Encyclopedia of industrial chemistry, Wiley-VCH, 2000, sections “dyes and pigments” and “dyes, general survey” (ISBN: 9783527303854)
Ullmann's Encyclopedia of Industrial Chemistry, Wiley-VCH Verlag Gmbh & Co. KGaA. 2018, section “Polyesters”, 10.1002/14356007.a21_227.pub2
“Industrial Organic Chemistry”, 3. volume, Wiley-VCH, 1997, ISBN: 978-3-527-28838-0,
“Kunststoffhandbuch”, Carl Hanser Verlag; vol. 6, “Polyamide”, 1. edition, 1966,
Kunststoffhandbuch”, Carl Hanser Verlag; vol. 7, “Polyurethane”, 3. edition, 1993,
Kunststoffhandbuch”, Carl Hanser Verlag; vol. 8, “Polyester”, 2. edition 1973;
“Industrial Organic Chemistry”, 3. volume, Wiley-VCH, 1997, ISBN: 978-3-527-28838-0,
“Injection Molding Reference Guide, 4th edition, CreateSpace Independent Publishing Platform, 2011, ISBN: 978-1466407824,
EP 0989146 A1
EP 1460094 A1
WO 2006034800 A1
EP 1529792 A1
WO 2006042674 A1
EP 0364854 A2
U.S. Pat. No. 5,506,275 A
EP 0897402 A1
WO 2015082316 A1
WO 2021021855 A1
WO 2021126938 A1
WO 2021021902 A1
WO 2021092311 A1
WO 2008155271 A1
WO 2013139827 A1
US 2016/0009893 A1

1. A process for recycling of a colored polymeric material, wherein the colored polymeric material comprises (i) a polyalkylene terephthalate based polymer, (ii) a colorant, and optionally (iii) a second polymer different from the polyalkylene terephthalate based polymer of (i), and optionally (iv) a third polymer different from the polyalkylene terephthalate based polymer of (i) and different from the second polymer of (iii), the process comprising
(a) providing a colored polymeric material and a solvent system comprising at least 90 weight-% gamma valerolactone (GVL);
(b) contacting the colored polymeric material provided in (a) with the solvent system comprising GVL provided in (a), under conditions allowing for colorant removal but not allowing for dissolution of the polyalkylene terephthalate based polymer, thereby obtaining a solvent system, which is enriched in colorant compared to the solvent system provided, and a polymeric material, which is depleted in colorant compared to the colored polymeric material provided in (a); and
(c) depolymerizing or transesterifying the polymeric material, which is depleted in colorant obtained in (b); thereby obtaining monomers, and/or oligomers, or transesterification products;
wherein (b) comprises
(b.1) contacting the colored polymeric material of (a) with the solvent system at a temperature T, which is in the range of from 110 to 165° C., thereby obtaining a solvent system, which is enriched in colorant and optionally in third polymer (iv) compared to the solvent system provided in (a), and a polymeric material, which is depleted in colorant and optionally in third polymer (iv) compared to the colored polymeric material provided in (a); and
(b.2) optionally separating the polymeric material, which is depleted in colorant and optionally in third polymer (iv), and which comprises the polyalkylene terephthalate based polymer, from the solvent system.

2. The process according to claim 1, wherein at least 95 weight-% of the solvent system consist of GVL.

3. The process according to claim 1, wherein the depolymerisation of (c) is done by a method selected from the group consisting of alcoholysis, glycolysis, hydrolysis, aminolysis, ammonolysis, and mixtures of two or more of these methods.

4. The process according to claim 1, wherein the transesterification of (c) is done by acid or base catalyzed reaction with an alcohol.

5. The process according to claim 1, wherein the second polymer (iii) is selected from the group consisting of polypropylene (PP), polyethylene (PE), polyamide (PA), and natural polymer; and/or wherein the third polymer (iv) is an elastic fiber.

6. The process according to claim 1, wherein the colorant is selected from the group consisting of dye, optical brightener, and mixtures of dye and optical brightener.

7. The process according to claim 1, wherein the polyalkylene terephthalate based polymer comprises or is polyethylene terephthalate (PET).

8. A monomer oligomer, or transesterification product of the polyalkylene terephthalate based polymer obtained by the process according to claim 1.

9. The process according to claim 1, comprising the further step of converting the monomers, oligomers, or transesterification products of the polyalkylene terephthalate based polymer obtained according to claim 1 to obtain one or more polymer or polymer product.

10. The process according to claim 1, comprising the further step of converting the separated second polymer (iii) and/or the separated third polymer (iv) to obtain one or more monomer, polymer, or polymer product.

11. The process according to claim 10, wherein the polymer is and/or the polymer product comprises polyamide (PA), polyisocyanate polyaddition product, low-density polyethylene (LDPE), high-density polyethylene (HDPE), polyethylene (PE), polypropylene (PP), polyvinyl chloride (PVC), polyvinyl acetate (PVA), polystyrene (PS), poly acrylonitrile butadiene styrene (ABS), poly styrene acrylonitrile (SAN), poly acrylate styrene acrylonitrile (ASA), polytetrafluoroethylene (PTFE), poly(methyl acrylate) (PMA), poly(methyl methacrylate) (PMMA), polybutadiene (BR, PBD), poly(cis-1,4-isoprene), poly(trans-1,4-isoprene), polyoxymethylene (POM), polyethylene terephthalate (PET), polybutylene terephthalate (PBT), polybutylene adipate coterephthalate (PBAT), polyester (PES), poly-ether sulfone (PESU), polyhydroxyalkanoate (PHA), poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), polylactic acid (PLA), polysulfone (PSU), polyphenylene sulfone (PPSU), polycarbonate (PC), polyether ether ketone (PEEK), poly(p-phenylene oxide) (PPO), poly(p-phenylene ether) (PPE); or copolymer, or a mixture thereof.

12. (canceled)

13. (canceled)

14. The process according to claim 1, wherein at least 98 weight-%, of the solvent system consist of GVL.

15. The process according to claim 1, wherein the second polymer (iii) is selected from the group consisting of polypropylene (PP), polyethylene (PE), polyamide (PA), PA6 PA66, natural polymer, cotton, viscose, linen, and mixtures of two or more thereof.

16. The process according to claim 1, wherein the third polymer (iv) is an elastic fiber, which comprises or consists of one or more polyurethane based elastic fiber(s) and/or one or more polyester based elastic fiber(s).

17. The process according to claim 1, wherein the third polymer (iv) is an elastic fiber, wherein the elastic fiber comprises or consists of one or more polyurethane based elastic fiber(s).

18. The process according to claim 1, wherein the third polymer (iv) is an elastic fiber, wherein at least 40 weight-% of the elastic fibers are polyurethane based elastic fibers.

19. The process according to claim 1, comprising the further step of polymerizing the monomers, oligomers, or transesterification products of the polyalkylene terephthalate based polymer obtained from the process according to claim 1, to obtain one or more polymer or polymer product; wherein the one or more polymer is/are polyalkylene terephthalate based polymer and the polymer product is a textile, a fiber, a packaging, or a plastic.

20. The process according to claim 16, wherein the polymer product is/are or is/are a part of:
a part of a car;
a cloth;
an electrical part;
a consumer, agricultural product or pharmaceutical product;
a packaging for the food industry; or
a part of a construction.

21. The process according to claim 16, wherein the polymer product is/are or is/are a part of:
a part of a car selected from the group consisting of cylinder head covers, engine covers, housings for charge air coolers, charge air cooler flaps, intake pipes, intake manifolds, connectors, gear wheels, fan wheels, cooling water boxes, housings, housing parts for heat exchangers, coolant coolers, charge air coolers, thermostats, water pumps, radiators, fastening parts, parts of battery systems for electromobility, dashboards, steering column switches, seats, headrests, center consoles, transmission components, door modules, A, B, C or D pillar covers, spoilers, door handles, exterior mirrors, windscreen wipers, windscreen wiper protection housings, decorative grills, cover strips, roof rails, window frames, sunroof frames, antenna panels, headlights and taillights, engine covers, cylinder head covers, intake manifolds, airbags, cushions, and coatings;
a cloth selected from the group consisting of shirts, trousers, pullovers, boots, shoes, shoe soles, tights, and jackets;
an electrical part selected from the group consisting of electrical or electronic passive or active components, circuit boards, printed circuit boards, housing components, foils, lines, switches, plugs, sockets, distributors, relays, resistors, capacitors, inductors, bobbins, lamps, diodes, LEDs, transistors, connectors, regulators, integrated circuits (IC), processors, controllers, memories, sensors, microswitches, microbuttons, semiconductors, reflector housing for light-emitting diodes (LED), fasteners for electrical or electronic components, spacers, bolts, strips, slide-in guides, screws, nuts, film hinges, snap hooks (snap-ins), and spring tongues;
a consumer, agricultural product or pharmaceutical product selected from the group consisting of tennis strings, climbing ropes, bristles, brushes, artificial grass, 3D printing filaments, grass trimmers, zippers, hooks and loop fasteners, paper machine clothing, extrusion coating, fishing lines, fishing nets, offshore lines and ropes, vials, syringes, ampoules, bottles, sliding elements, spindle nuts, chain conveyors, plain bearings, rollers, wheels, gears, rollers, ring gears, screws and spring dampers, hoses, pipelines, cable sheathing, sockets, switches, cable ties, fan wheels, carpets, box or bottle for cosmetics, mattresses, cushions, insulations, detergents, dishwasher tabs or powders, shampoos, body washes, shower gels, soaps, fertilizers, fungicides, and pesticides;
a packaging for the food industry selected from the group consisting of mono- or multilayer blown films, cast films (mono- or multi-layer), biaxially stretched films, and laminating films; or
a part of a construction selected from the group consisting of rotor blades, insulating materials, frames, housings, walls, coatings, and separating walls.

22. The process according to claim 10, wherein the polymer is and/or the polymer product comprises PA 6, PA 66, polyurethane (PU), thermoplastic polyurethane (TPU), polyurea or polyisocyanurate (PIR); or copolymers or mixtures thereof.
